(19) United States
(12) Patent Application Publication
(10) Pub. No.: US 2014/0370104 A1
(43) Pub. Date: Dec. 18, 2014

(54) PHARMACEUTICAL FORMULATION AND METHOD FOR TREATING ACID CAUSED GASTROINTESTINAL DISORDERS

(71) Applicant: Santarus, Inc., San Diego, CA (US)

(72) Inventors: Warren HALL, Del Mar, CA (US); Kay OLMSTEAD, San Diego, CA (US); Laura WESTON, Escondido, CA (US)

(73) Assignee: Santarus, Inc., San Diego, CA (US)

(21) Appl. No.: 14/181,017

(22) Filed: Feb. 14, 2014

Related U.S. Application Data
(63) Continuation of application No. 12/847,938, filed on Jul. 30, 2010, now abandoned.
Application No. 12/847,938 is a continuation of application No. 10/893,092, filed on Jul. 16, 2004, now abandoned.
(60) Provisional application No. 60/488,324, filed on Jul. 18, 2003.

(51) Int. Cl. A61K 47/02; A61K 31/4439; A61K 9/14

(52) U.S. Cl. CPC A61K 47/02; A61K 9/14; A61K 31/4439
USPC 424/490; 514/338

(57) ABSTRACT

Pharmaceutical formulations in the form of a powder for suspension comprising at least one proton pump inhibitor in micronized form; at least one antacid; and at least one suspending agents are provided herein. Also provided herein are methods for making and using pharmaceutical formulations comprising at least one proton pump inhibitor and at least one antacid.

This application is a continuation application of U.S. application Ser. No. 12/847,938 filed Jul. 30, 2010, which is a continuation application of and claims priority to U.S. application Ser. No. 10/893,092, filed Jul. 16, 2004, now abandoned, which claims the benefit of U.S. Provisional Application No. 60/488,324, filed Jul. 18, 2003, the contents of all applications are fully incorporated by reference herewith.

### FIELD OF THE INVENTION

The present invention relates to pharmaceutical formulations comprising a proton pump inhibitor, at least one antacid, and at least one suspending agent. In addition, methods for manufacture of the pharmaceutical formulations; uses of the pharmaceutical formulations in treating disease; and combinations of the pharmaceutical formulations with other therapeutic agents are described.

### BACKGROUND OF THE INVENTION

Upon ingestion, most acid-labile pharmaceutical compounds must be protected from contact with acidic stomach secretions to maintain their pharmaceutical activity. To accomplish this, compositions with enteric-coatings have been designed to dissolve at a pH to ensure that the drug is released in the proximal region of the small intestine (duodenum), rather than the acidic environment of the stomach. However, due to the pH-dependent attributes of these enteric-coated compositions and the uncertainty of gastric retention time, in-vivo performance as well as both inter- and infra-subject variability are all major set backs of using enteric-coated systems for the controlled release of a drug.

In addition, Phillips et al. has described non-enteric coated pharmaceutical compositions. These compositions, which allow for the immediate release of the pharmaceutically active ingredient into the stomach, involve the administration of one or more buffering agents with an acid labile pharmaceutical agent, such as a proton pump inhibitor. The buffering agent is thought to prevent substantial degradation of the acid labile pharmaceutical agent in the acidic environment of the stomach by raising the pH. See, e.g., U.S. Pat. Nos. 5,840,737; 6,489,346; 6,645,988; and 6,699,885.

A class of acid-labile pharmaceutical compounds that are administered as enteric-coated dosage forms are proton pump inhibiting agents. Exemplary proton pump inhibitors include, omeprazole (Prilosec®), lansoprazole (Prevacid®), esomeprazole) (Nexium®), rabeprazole (Aciphex®), pantoprazole (Protonix®), pariprazole, tentaprazole, and leminoprazole. The drugs of this class suppress gastrointestinal acid secretion by the specific inhibition of the H⁺/K⁺-ATPase enzyme system (proton pump) at the secretory surface of the gastrointestinal parietal cell. Most proton pump inhibitors are susceptible to acid degradation and, as such, are rapidly destroyed as pH falls to an acidic level. Therefore, if the enteric-coating of these formulated products is disrupted (e.g., trituration to compound a liquid, or chewing the capsule or tablet) or the buffering agent fails to sufficiently neutralize the gastrointestinal pH, the drug will be exposed to degradation by the gastrointestinal acid in the stomach.

Omeprazole is one example of a proton pump inhibitor which is a substituted bicyclic aryl-imidazole, 5-methoxy-2-[(4-methoxy-3,5-dimethyl-2-pyridinyl)methyl]sulfinyl]-1H-benzimidazole, that inhibits gastrointestinal acid secretion. U.S. Pat. No. 4,786,505 to Lovgren et al. teaches that a pharmaceutical oral solid dosage form of omeprazole must be protected from contact with acidic gastrointestinal juice by an enteric-coating to maintain its pharmaceutical activity and describes an enteric-coated omeprazole preparation containing one or more subcoats between the core material and the enteric-coating.

Proton pump inhibitors are typically prescribed for short-term treatment of active duodenal ulcers, gastrointestinal ulcers, gastro esophageal reflux disease (GERD), severe erosive esophagitis, poorly responsive symptomatic GERD, and pathological hypersecretory conditions such as Zollinger Ellison syndrome. These above-listed conditions commonly arise in healthy or critically ill patients of all ages, and may be accompanied by significant upper gastrointestinal bleeding.

It is believed that omeprazole, lansoprazole and other proton pump inhibiting agents reduce gastrointestinal acid production by inhibiting H⁺/K⁺-ATPase of the parietal cell the final common pathway for gastrointestinal acid secretion. See, e.g., Fellenius et al., Substituted Benzimidazoles Inhibit Gastrointestinal Acid Secretion by Blocking H⁺/K⁺-ATPase, Nature, 290: 159-161(1981); Wallmark et al., The Relationship Between Gastrointestinal Acid Secretion and Gastrointestinal H⁺/K⁺-ATPase Activity, J. Biol. Chem., 260: 13681-13684 (1985); and Fryklund et al., Function and Structure of Parietal Cells After H⁺/K⁺-ATPase Blockade, Am. J. Physiol., 254 (1988).

Proton pump inhibitors have the ability to act as weak bases which reach parietal cells from the blood and diffuse into the secretory canaliculi. There the drugs become protonated and thereby trapped. The protonated compound can then rearrange to form a sulfenamide which can covalently interact with sulfhydryl groups at critical sites in the extra cellular (luminal) domain of the membrane-spanning H⁺/K⁺-ATPase. See, e.g., Hardman et al., Goodman & Gilman's The Pharmacological Basis of Therapeutics, 907 (9th ed. 1996). As such, proton pump inhibitors are prodrugs that must be activated to be effective. The specificity of the effects of proton pump inhibiting agents is also dependent upon: (a) the selective distribution of H⁺/K⁺-ATPase; (b) the requirement for acidic conditions to catalyze generation of the reactive inhibitor; and (c) the trapping of the protonated drug and the cationic sulfenamide within the acidic canaliculi and adjacent to the target enzyme. See, e.g., Hardman et al.

Thus, there remains a need for a pharmaceutical formulation that can be administered in a stable, uniform suspension whereby the proton pump inhibitor is released in the stomach. In addition, for patient compliance, a need remains for an improved formulation which masks the bitter taste of the proton pump inhibitor and other excipients to provide a more palatable formulation.

### SUMMARY OF THE INVENTION

The present invention relates to pharmaceutical formulations comprising at least one proton pump inhibiting agent, at least one antacid and at least one suspending agent that have been found to possess improved suspendability, bioavailability, chemical stability, physical stability, dissolution profiles, disintegration times, safety, as well as other improved pharmacokinetic, pharmacodynamic, chemical and/or physical properties. The pharmaceutical formulations of the present invention are useful for administration of a suspension to a subject.

Pharmaceutical formulations in the form of a powder for suspension comprising at least one proton pump inhibitor in a micronized form; at least one antacid; at least one suspending agent; wherein a substantially uniform suspension is obtained upon admixture with water are provided herein.

Also provided herein are pharmaceutical formulations in the form of a powder for suspension comprising at least one proton pump inhibitor in a micronized form; at least one antacid; and a suspending agent wherein the suspending agent is a gum; and wherein upon admixture with water, a first suspension is obtained that is substantially more uniform when compared to a second suspension comprising the proton pump inhibitor, the antacid, the flavoring agent, and a suspending agent, wherein the suspending agent is not a gum, are described.

Pharmaceutical formulation comprising: (a) at least one acid-labile proton pump inhibitor in micronized form; and (b) at least one antacid, wherein the pharmaceutical formulation is made by a method comprising the steps of: (a) coating at least some of the at least one antacid with at least some of the micronized proton pump inhibitor to form a first blend; and (b) dry-blending the first blend with at least one other excipient are provided herein.

Also provided herein are methods of treating a condition or disorder by administering a pharmaceutical formulation of the invention where treatment with an inhibitor of H⁺/K⁺-ATPase is indicated, such as an acid-caused gastrointestinal disorder.

### BRIEF DESCRIPTION OF THE DRAWINGS

FIG. 1 is a SEM photo of sodium bicarbonate coated with micronized omeprazole.

FIG. 2 is a SEM photo of sodium bicarbonate.

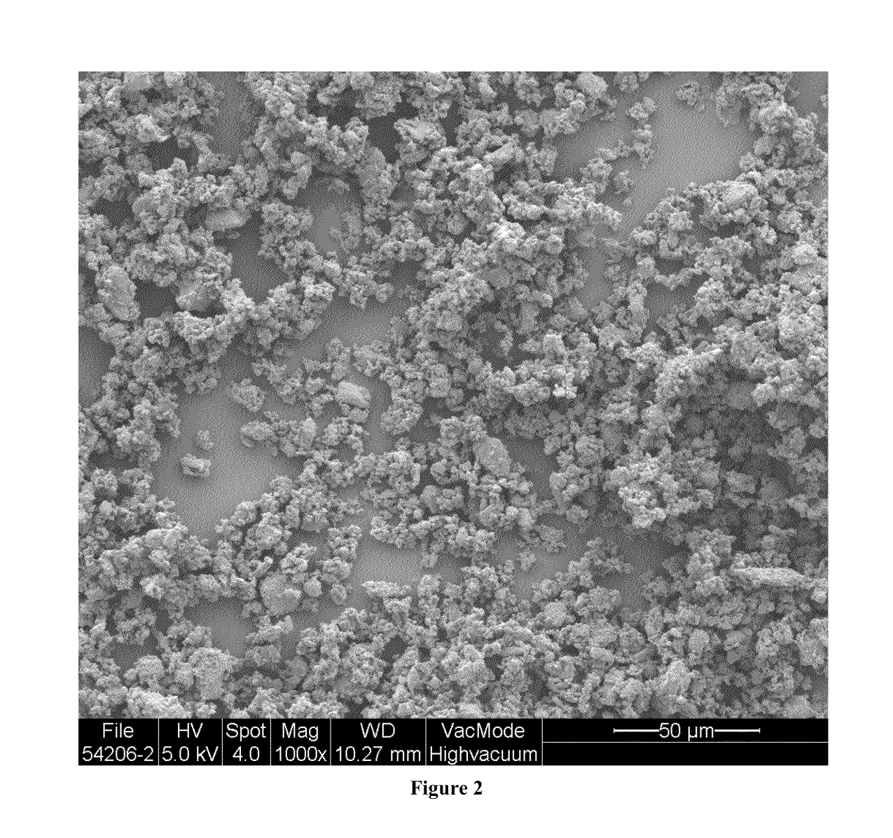

FIG. 3 is a SEM photo of micronized omeprazole.

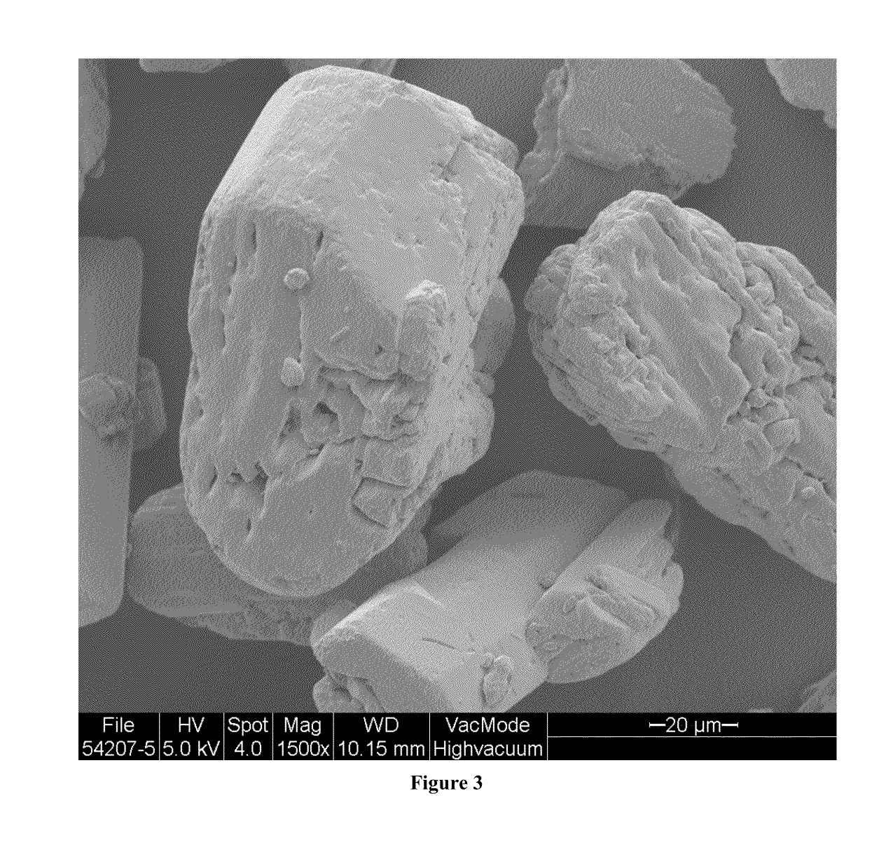

### DETAILED DESCRIPTION OF THE INVENTION

The present invention provides pharmaceutical formulations for administration of suspension comprising at least one proton pump inhibiting agent, at least one antacid, at least one suspending agent; and at least one flavoring agent.

The present invention is also directed to methods of treating a condition or disorder by administering a pharmaceutical formulation of the invention where treatment with an inhibitor of H+, K+-ATPase is indicated, such as an acid-caused gastrointestinal disorder.

While the present invention may be embodied in many different forms, several specific embodiments are discussed herein with the understanding that the present disclosure is to be considered only as an exemplification of the principles of the invention, and it is not intended to limit the invention to the embodiments illustrated.

To more readily facilitate an understanding of the invention and its preferred embodiments, the meanings of terms used herein will become apparent from the context of this specification in view of common usage of various terms and the explicit definitions of other terms provided in the glossary below or in the ensuing descriptions.

### GLOSSARY

As used herein, the terms “comprising,” “including,” and “such as” are used in their open, non-limiting sense.

The term “about” is used synonymously with the term “approximately.” Illustratively, the use of the term “about” indicates that values slightly outside the cited values, i.e., plus or minus 0.1% to 10%, which are also effective and safe. Such dosages are thus encompassed by the scope of the claims reciting the terms “about” and “approximately.”

The phrase “acid-labile pharmaceutical agent” refers to any pharmacologically active drug subject to acid catalyzed degradation.

“Aftertaste” is a measurement of all sensation remaining after swallowing. Aftertaste can be measured, e.g., from 30 seconds after swallowing, 1 minute after swallowing, 2 minutes after swallowing, 3 minutes after swallowing, 4 minutes after swallowing, 5 minutes after swallowing, and the like.

“Amplitude” is the initial overall perception of the flavors balance and fullness. The amplitude scale is 0-none, 1-low, 2-moderate, and 3-high.

“Anti-adherents,” “glidants,” or “anti-adhesion” agents prevent components of the formulation from aggregating or sticking and improve flow characteristics of a material. Such compounds include, e.g., colloidal silicon dioxide such as Cab-o-sil®; tribasic calcium phosphate, talc, corn starch, DL-leucine, sodium lauryl sulfate, magnesium stearate, calcium stearate, sodium stearate, kaolin, and micronized amorphous silicon dioxide (Syloid®) and the like.

“Antifoaming agents” reduce foaming during processing which can result in coagulation of aqueous dispersions, bubbles in the finished film, or generally impair processing. Exemplary anti-foaming agents include silicon emulsions or sorbitan sesquoleate.

“Antioxidants” include, e.g., butylated hydroxytoluene (BHT), butylated hydroxyanisole (BHA), sodium ascorbate, and tocopherol.

“Binders” impart cohesive qualities and include, e.g., alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crospovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

“Bioavailability” refers to the extent to which an active moiety, e.g., drug, prodrug, or metabolite, is absorbed into the general circulation and becomes available at the site of drug action in the body. Thus, a proton pump inhibitor administered through IV is 100% bioavailable. “Oral bioavailability” refers to the extent to with the proton pump inhibitor is absorbed into the general circulation and becomes available at the site of the drug action in the body when the pharmaceutical formulation is taken orally.

“Bioequivalence” or “bioequivalent” means that the area under the serum concentration time curve (AUC) and the peak serum concentration (Cₘₐₓ) are each within 80% and 125%.

“Carrier materials” include any commonly used excipients in pharmaceutics and should be selected on the basis of compatibility with the proton pump inhibitor and the release profile properties of the desired dosage form. Exemplary carrier materials include, e.g., binders, suspending agents, disintegration agents, filling agents, surfactants, solubilizers, stabilizers, lubricants, wetting agents, diluents, and the like. “Pharmaceutically compatible carrier materials” may comprise, e.g., acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

“Character notes” include, e.g., aromatics, basis tastes, and feeling factors. The intensity of the character note can be scaled from 0-none, 1-slight, 2-moderate, or 3-strong.

A “derivative” is a compound that is produced from another compound of similar structure by the replacement of substitution of an atom, molecule or group by another suitable atom, molecule or group. For example, one or more hydrogen atom of a compound may be substituted by one or more alkyl, acyl, amino, hydroxyl, halo, haloalkyl, aryl, heteroaryl, cycloaolkyl, heterocycloalkyl, or heteroalkyl group to produce a derivative of that compound.

“Diffusion facilitators” and “dispersing agents” include materials that control the diffusion of an aqueous fluid through a coating. Exemplary diffusion facilitators/dispersing agents include, e.g., hydrophilic polymers, electrolytes, Tween® 60 or 80, PEG and the like. Combinations of one or more erosion facilitator with one or more diffusion facilitator can also be used in the present invention.

“Diluents” increase bulk of the composition to facilitate compression. Such compounds include e.g., lactose; starch; mannitol; sorbitol; dextrose; microcrystalline cellulose such as Avicel®; dibasic calcium phosphate; dicalcium phosphate dihydrate; tricalcium phosphate; calcium phosphate; anhydrous lactose; spray-dried lactose; pregelatinzed starch; compressible sugar, such as Di-Pac® (Amstar); mannitol; hydroxypropylmethylcellulose; sucrose-based diluents; confectioner's sugar; monobasic calcium sulfate monohydrate; calcium sulfate dihydrate; calcium lactate trihydrate; dextrates; hydrolyzed cereal solids; amylose; powdered cellulose; calcium carbonate; glycine; kaolin; mannitol; sodium chloride; inositol; bentonite; and the like.

The term “disintegrate” includes both the dissolution and dispersion of the dosage form when contacted with gastrointestinal fluid.

“Disintegration agents” facilitate the breakup or disintegration of a substance. Examples of disintegration agents include a starch, e.g., a natural starch such as corn starch or potato starch, a pregelatinized starch such as National 1551 or Amijel®, or sodium starch glycolate such as Promogel® or Explotab®; a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

“Drug absorption” or “absorption” refers to the process of movement from the site of administration of a drug toward the systemic circulation, e.g., into the bloodstream of a subject.

An “enteric coating” is a substance that remains substantially intact in the stomach but dissolves and releases the drug once the small intestine is reached. Generally, the enteric coating comprises a polymeric material that prevents release in the low pH environment of the stomach but that ionizes at a slightly higher pH, typically a pH of 4 or 5, and thus dissolves sufficiently in the small intestines to gradually release the active agent therein.

The “enteric form of the proton pump inhibitor” is intended to mean that some or most of the proton pump inhibitor has been enterically coated to ensure that at least some of the drug is released in the proximal region of the small intestine (duodenum), rather than the acidic environment of the stomach.

“Erosion facilitators” include materials that control the erosion of a particular material in gastrointestinal fluid. Erosion facilitators are generally known to those of ordinary skill in the art. Exemplary erosion facilitators include, e.g., hydrophilic polymers, electrolytes, proteins, peptides, and amino acids.

“Filling agents” include compounds such as lactose, calcium carbonate, calcium phosphate, dibasic calcium phosphate, calcium sulfate, microcrystalline cellulose, cellulose powder, dextrose; dextrates; dextran, starches, pregelatinized starch, sucrose, xylitol, lactitol, mannitol, sorbitol, sodium chloride, polyethylene glycol, and the like.

“Flavoring agents” or “sweeteners” useful in the pharmaceutical compositions of the present invention include, e.g., acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

“Gastrointestinal fluid” is the fluid of stomach secretions of a subject or the saliva of a subject after oral administration of a composition of the present invention, or the equivalent thereof. An “equivalent of stomach secretion” includes, e.g., an in vitro fluid having similar content and/or pH as stomach secretions such as a 1% sodium dodecyl sulfate solution or 0.1N HCl solution in water.

“Half-life” refers to the time required for the plasma drug concentration or the amount in the body to decrease by 50% from its maximum concentration.

“Lubricants” are compounds that prevent, reduce or inhibit adhesion or friction of materials. Exemplary lubricants include, e.g., stearic acid; calcium hydroxide; talc; sodium stearyl fumerate; a hydrocarbon such as mineral oil, or hydrogenated vegetable oil such as hydrogenated soybean oil (Sterotex®); higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol or a methoxypolyethylene glycol such as Carbowax™, sodium oleate, glyceryl behenate, polyethylene glycol, magnesium or sodium lauryl sulfate, colloidal silica such as Syloid™, Carb-O-Sil®, a starch such as corn starch, silicone oil, a surfactant, and the like.

A “measurable serum concentration” or “measurable plasma concentration” describes the blood serum or blood plasma concentration, typically measured in mg, μg, or ng of therapeutic agent per ml, dl, or l of blood serum, of a therapeutic agent that is absorbed into the bloodstream after administration. One of ordinary skill in the art would be able to measure the serum concentration or plasma concentration of a proton pump inhibitor or a prokinetic agent. See, e.g., Gonzalez H. et al., J. Chromatogr. B. Analyt. Technol. Biomed. Life Sci., vol. 780, pp 459-65, (Nov. 25, 2002).

“Parietal cell activators” or “activators” stimulate the parietal cells and enhance the pharmaceutical activity of the proton pump inhibitor. Parietal cell activators include, e.g., chocolate; alkaline substances such as sodium bicarbonate; calcium such as calcium carbonate, calcium gluconate, calcium hydroxide, calcium acetate and calcium glycerophosphate; peppermint oil; spearmint oil; coffee; tea and colas (even if decaffeinated); caffeine; theophylline; theobromine; amino acids (particularly aromatic amino acids such as phenylalanine and tryptophan); and combinations thereof.

“Pharmacodynamics” refers to the factors that determine the biologic response observed relative to the concentration of drug at a site of action.

“Pharmacokinetics” refers to the factors that determine the attainment and maintenance of the appropriate concentration of drug at a site of action.

“Plasma concentration” refers to the concentration of a substance in blood plasma or blood serum of a subject. It is understood that the plasma concentration of a therapeutic agent may vary many-fold between subjects, due to variability with respect to metabolism of therapeutic agents. In accordance with one aspect of the present invention, the plasma concentration of a proton pump inhibitors and/or prokinetic agent may vary from subject to subject. Likewise, values such as maximum plasma concentration (Cₘₐₓ) or time to reach maximum serum concentration (Tₘₐₓ), or area under the serum concentration time curve (AUC) may vary from subject to subject. Due to this variability, the amount necessary to constitute “a therapeutically effective amount” of proton pump inhibitor, prokinetic agent, or other therapeutic agent, may vary from subject to subject. It is understood that when mean plasma concentrations are disclosed for a population of subjects, these mean values may include substantial variation.

“Plasticizers” are compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, and triacetin.

“Prevent” or “prevention” when used in the context of a gastric acid related disorder means no gastrointestinal disorder or disease development if none had occurred, or no further gastrointestinal disorder or disease development if there had already been development of the gastrointestinal disorder or disease. Also considered is the ability of one to prevent some or all of the symptoms associated with the gastrointestinal disorder or disease.

A “prodrug” refers to a drug or compound in which the pharmacological action results from conversion by metabolic processes within the body. Prodrugs are generally drug precursors that, following administration to a subject and subsequent absorption, are converted to an active, or a more active species via some process, such as conversion by a metabolic pathway. Some prodrugs have a chemical group present on the prodrug that renders it less active and/or confers solubility or some other property to the drug. Once the chemical group has been cleaved and/or modified from the prodrug the active drug is generated. Prodrugs may be designed as reversible drug derivatives, for use as modifiers to enhance drug transport to site-specific tissues. The design of prodrugs to date has been to increase the effective water solubility of the therapeutic compound for targeting to regions where water is the principal solvent. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987.

“Proton pump inhibitor product” refers to a product sold on the market. Proton pump inhibitor products include, for example, Priolosec®, Nexium®, Prevacid®, Protonic®, and Aciphex®.

“Serum concentration” refers to the concentration of a substance such as a therapeutic agent, in blood plasma or blood serum of a subject. It is understood that the serum concentration of a therapeutic agent may vary many-fold between subjects, due to variability with respect to metabolism of therapeutic agents. In accordance with one aspect of the present invention, the serum concentration of a proton pump inhibitors and/or prokinetic agent may vary from subject to subject. Likewise, values such as maximum serum concentration (Cₘₐₓ) or time to reach maximum serum concentration (Tₘₐₓ), or total area under the serum concentration time curve (AUC) may vary from subject to subject. Due to this variability, the amount necessary to constitute “a therapeutically effective amount” of proton pump inhibitor, prokinetic agent, or other therapeutic agent, may vary from subject to subject. It is understood that when mean serum concentrations are disclosed for a population of subjects, these mean values may include substantial variation.

“Solubilizers” include compounds such as citric acid, succinic acid, fumaric acid, malic acid, tartaric acid, maleic acid, glutaric acid, sodium bicarbonate, sodium carbonate and the like.

“Stabilizers” include compounds such as any antioxidation agents, buffers, acids, and the like.

“Suspending agents” or “thickening agents” include compounds such as polyvinylpyrrolidone, e.g., polyvinylpyrrolidone K12, polyvinylpyrrolidone K17, polyvinylpyrrolidone K25, or polyvinylpyrrolidone K30; polyethylene glycol, e.g., the polyethylene glycol can have a molecular weight of about 300 to about 6000, or about 3350 to about 4000, or about 7000 to about 5400; sodium carboxymethylcellulose; methylcellulose; hydroxy-propylmethylcellulose; polysorbate-80; hydroxyethylcellulose; sodium alginate; gums, such as, e.g., gum tragacanth and gum acacia; guar gum; xanthans, including xanthan gum; sugars; cellulosics, such as, e.g., sodium carboxymethylcellulose, methylcellulose, sodium carboxymethylcellulose, hydroxypropylmethylcellulose, hydroxyethylcellulose; polysorbate-80; sodium alginate; polyethoxylated sorbitan monolaurate; polyethoxylated sorbitan monolaurate; povidone and the like.

“Surfactants” include compounds such as sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF); and the like.

A “therapeutically effective amount” or “effective amount” is that amount of a pharmaceutical agent to achieve a pharmacological effect. The term “therapeutically effective amount” includes, for example, a prophylactically effective amount. An “effective amount” of a proton pump inhibitor is an amount effective to achieve a desired pharmacologic effect or therapeutic improvement without undue adverse side effects. For example, an effective amount of a proton pump inhibitor refers to an amount of proton pump inhibitor that reduces acid secretion, or raises gastrointestinal fluid pH, or reduces gastrointestinal bleeding, or reduces the need for blood transfusion, or improves survival rate, or provides for a more rapid recovery from a gastric acid related disorder. The effective amount of a pharmaceutical agent will be selected by those skilled in the art depending on the particular patient and the disease level. It is understood that “an effect amount” or “a therapeutically effective amount” can vary from subject to subject, due to variation in metabolism of therapeutic agents such as proton pump inhibitors and/or prokinetic agents, age, weight, general condition of the subject, the condition being treated, the severity of the condition being treated, and the judgment of the prescribing physician.

“Total intensity of aroma” is the overall immediate impression of the strength of the aroma and includes both aromatics and nose feel sensations.

“Total intensity of flavor” is the overall immediate impression of the strength of the flavor including aromatics, basic tastes and mouth feel sensations.

“Treat” or “treatment” as used in the context of a gastric acid related disorder refers to any treatment of a disorder or disease associated with a gastrointestinal disorder, such as preventing the disorder or disease from occurring in a subject which may be predisposed to the disorder or disease, but has not yet been diagnosed as having the disorder or disease; inhibiting the disorder or disease, e.g., arresting the development of the disorder or disease, relieving the disorder or disease, causing regression of the disorder or disease, relieving a condition caused by the disease or disorder, or stopping the symptoms of the disease or disorder. Thus, as used herein, the term “treat” is used synonymously with the term “prevent.”

“Wetting agents” include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, and the like.

### Proton Pump Inhibitors

The terms “proton pump inhibitor,” “PPI,” and “proton pump inhibiting agent” can be used interchangeably to describe any acid labile pharmaceutical agent possessing pharmacological activity as an inhibitor of H+/K+-ATPase. A proton pump inhibitor may, if desired, be in the form of free base, free acid, salt, ester, hydrate, anhydrate, amide, enantiomer, isomer, tautomer, prodrug, polymorph, derivative, or the like, provided that the free base, salt, ester, hydrate, amide, enantiomer, isomer, tautomer, prodrug, or any other pharmacologically suitable derivative is therapeutically active.

Proton pump inhibitors can be a substituted bicyclic aryl-imidazole, wherein the aryl group can be, e.g., a pyridine, a phenyl, or a pyrimidine group and is attached to the 4- and 5-positions of the imidazole ring. Proton pump inhibitors comprising a substituted bicyclic aryl-imidazoles include, e.g., omeprazole, hydroxyomeprazole, esomeprazole, lansoprazole, pantoprazole, rabeprazole, dontoprazole, habeprazole, periprazole, tenatoprazole, ransoprazole, pariprazole, leminoprazole, or a free base, free acid, salt, hydrate, ester, amide, enantiomer, isomer, tautomer, polymorph, prodrug, or derivative thereof. See, e.g., The Merck Index, Merck & Co. Rahway, N.J. (2001).

Other proton pump inhibitors include, e.g., soraprazan (Altana); ilaprazole (U.S. Pat. No. 5,703,097) (Il-Yang); AZD-0865 (AstraZeneca); YH-1885 (PCT Publication WO 96/05177) (SB-641257) (2-pyrimidinamine, 4-(3,4-dihydro-1-methyl-2(1H)-isoquinolinyl)-N-(4-fluorophenyl)-5,6-dimethyl-, monohydrochloride) (YuHan); BY-112 (Altana); SP1-447 (Imidazo(1,2-a)thieno(3,2-c)pyridin-3-amine, 5-methyl-2-(2-methyl-3-thienyl) (Shinnippon); 3-hydroxymethyl-2-methyl-9-phenyl-7H-8,9-dihydro-pyrano(2,3-c)-imidazo(1,2-a)pyridine (PCT Publication WO 95/27714) (AstraZeneca); Pharmaprojects No. 4950 (3-hydroxymethyl-2-methyl-9 phenyl-7H-8,9-dihydro-pyrano(2,3-c)-imidazo(1,2-a)pyridine) (AstraZeneca, ceased) WO 95/27714; Pharmaprojects No. 4891 (EP 700899) (Aventis); Pharmaprojects No. 4697 (PCT Publication WO 95/32959) (AstraZeneca); H-335/25 (AstraZeneca); T-330 (Saitama 335) (Pharmacological Research Lab); Pharmaprojects No. 3177 (Roche); BY-574 (Altana); Pharmaprojects No. 2870 (Pfizer); AU-1421 (EP 264883) (Merck); AU-2064 (Merck); AY-28200 (Wyeth); Pharmaprojects No. 2126 (Aventis); WY-26769 (Wyeth); pumaprazole (PCT Publication WO 96/05199) (Altana); YH-1238 (YuHan); Pharmaprojects No. 5648 (PCT Publication WO 97/32854) (Dainippon); BY-686 (Altana); YM-020 (Yamanouchi); GYKI-34655 (Ivax); FPL-65372 (Aventis); Pharmaprojects No. 3264 (EP 509974) (AstraZeneca); nepaprazole (Toa Eiyo); HN-11203 (Nycomed Pharma); OPC-22575; pumilacidin A (BMS); saviprazole (EP 234485) (Aventis); SKandF-95601 (GSK, discontinued); Pharmaprojects No. 2522 (EP 204215) (Pfizer); S-3337 (Aventis); RS-13232A (Roche); AU-1363 (Merck); SKandF-96067 (EP 259174) (Altana); SUN 8176 (Daiichi Phama); Ro-18-5362 (Roche); ufiprazole (EP 74341) (AstraZeneca); and Bay-p-1455 (Bayer); or a free base, free acid, salt, hydrate, ester, amide, enantiomer, isomer, tautomer, polymorph, prodrug, or derivative of these compounds.

Still other proton pump inhibitors include those described in U.S. Pat. Nos. 4,628,098; 4,689,333; 4,786,505; 4,853,230; 4,965,269; 5,021,433; 5,026,560; 5,045,321; 5,093,132; 5,430,042; 5,433,959; 5,576,025; 5,639,478; 5,703,110; 5,705,517; 5,708,017; 5,731,006; 5,824,339; 5,855,914; 5,879,708; 5,948,773; 6,017,560; 6,123,962; 6,187,340; 6,296,875; 6,319,904; 6,328,994; 4,255,431; 4,508,905; 4,636,499; 4,738,974; 5,690,960; 5,714,504; 5,753,265; 5,817,338; 6,093,734; 6,013,281; 6,136,344; 6,183,776; 6,328,994, 6,479,075; 6,559,167.

Other substituted bicyclic aryl-imidazole compounds as well as their salts, hydrates, esters, amides, enantiomers, isomers, tautomers, polymorphs, prodrugs, and derivatives may be prepared using standard procedures known to those skilled in the art of synthetic organic chemistry. See, e.g., March, Advanced Organic Chemistry: Reactions, Mechanisms and Structure, 4th Ed. (New York: Wiley-Interscience, 1992); Leonard et al., Advanced Practical Organic Chemistry, (1992); Howarth et al; Core Organic Chemistry (1998); and Weisermel et al., Industrial Organic Chemistry (2002).

“Pharmaceutically acceptable salts,” or “salts,” include, e.g., the salt of a proton pump inhibitor prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic, methanesulfonic, ethanesulfonic, benzenesulfonic, pantothenic, toluenesulfonic, 2-hydroxyethanesulfonic, sulfanilic, cyclohexylaminosulfonic, algenic, b-hydroxybutyric, galactaric and galacturonic acids.

Acid addition salts are prepared from the free base using conventional methodology involving reaction of the free base with a suitable acid. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like.

An acid addition salt is reconverted to the free base by treatment with a suitable base. Acid addition salts of the proton pump inhibitors can be halide salts, which are prepared using hydrochloric or hydrobromic acids. The basic salts include alkali metal salts, e.g., sodium salt and copper salt.

Salt forms of proton pump inhibiting agents include, e.g., a sodium salt form such as esomeprazole sodium, omeprazole sodium, rabeprazole sodium, pantoprazole sodium; or a magnesium salt form such as esomeprazole magnesium or omeprazole magnesium, described in U.S. Pat. No. 5,900,424; or a calcium salt form; or a potassium salt form such as the potassium salt of esomeprazole, described in U.S. patent application Ser. No. 02/019,8239 and U.S. Pat. No. 6,511,996. Other salts of esomeprazole are described in U.S. Pat. No. 4,738,974 and U.S. Pat. No. 6,369,085. Salt forms of pantoprazole and lansoprazole are discussed in U.S. Pat. Nos. 4,758,579 and 4,628,098, respectively.

Preparation of esters involves functionalizing a hydroxyl and/or carboxyl group present within the molecular structure of the drug. For example, the esters can be acyl-substituted derivatives of free alcohol groups, e.g., moieties derived from carboxylic acids of the formula RCOOR₁, where R₁ is a lower alkyl group. Esters can be reconverted to the free acids, if desired, by using conventional procedures such as hydrogenolysis or hydrolysis.

“Amides” may be prepared using techniques known to those skilled in the art or described in the pertinent literature. For example, amides may be prepared from esters, using suitable amine reactants, or they may be prepared from an anhydride or an acid chloride by reaction with an amine group such as ammonia or a lower alkyl amine.

“Tautomers” of substituted bicyclic aryl-imidazoles include, e.g., tautomers of omeprazole such as those described in U.S. Pat. Nos. 6,262,085; 6,262,086; 6,268,385; 6,312,723; 6,316,020; 6,326,384; 6,369,087; and 6,444,689; and U.S. Patent Publication No. 02/10156103.

An exemplary “isomer” of a substituted bicyclic aryl-imidazole is the isomer of omeprazole. See, e.g., Oishi et al., Acta Cryst. (1989), C45, 1921-1923; U.S. Pat. No. 6,150,380; U.S. Patent Publication No. 02/0156284; and PCT Publication No. WO 02/085889.

Exemplary “polymorphs” include, e.g., those described in PCT Publication No. WO 92/08716; and U.S. Pat. Nos. 4,045,563; 4,182,766; 4,508,905; 4,628,098; 4,636,499; 4,689,333; 4,758,579; 4,783,974; 4,786,505; 4,808,596; 4,853,230; 5,026,560; 5,013,743; 5,035,899; 5,045,321; 5,045,552; 5,093,132; 5,093,342; 5,433,959; 5,464,632; 5,536,735; 5,576,025; 5,599,794; 5,629,305; 5,639,478; 5,690,960; 5,703,110; 5,705,517; 5,714,504; 5,731,006; 5,879,708; 5,900,424; 5,948,773; 5,997,903; 6,017,560; 6,123,962; 6,147,103; 6,150,380; 6,166,213; 6,191,148; 5,187,340; 6,268,385; 6,262,086; 6,262,085; 6,296,875; 6,316,020; 6,328,994; 6,326,384; 6,369,085; 6,369,087; 6,380,234; 6,428,810; 6,444,689; and 6,462,0577.

A “derivative” is a compound that is produced from another compound of similar structure by the replacement of substitution of an atom, molecule or group by another suitable atom, molecule or group. For example, one or more hydrogen atom of a compound may be substituted by one or more alkyl, acyl, amino, hydroxyl, halo, haloalkyl, aryl, heteroaryl, cycloaolkyl, heterocycloalkyl, or heteroalkyl group to produce a derivative of that compound.

A “prodrug” refers to a drug or compound in which the pharmacological action results from conversion by metabolic processes within the body. Prodrugs are generally drug precursors that; following administration to a subject and subsequent absorption, are converted to an active, or a more active species via some process, such as conversion by a metabolic pathway. Some prodrugs have a chemical group present on the prodrug which renders it less active and/or confers solubility or some other property to the drug. Once the chemical group has been cleaved and/or modified from the prodrug the active drug is generated.

Prodrugs may be designed as reversible drug derivatives, for use as modifiers to enhance drug transport to site-specific tissues. The design of prodrugs to date has been to increase the effective water solubility of the therapeutic compound for targeting to regions where water is the principal solvent. See, e.g., Fedorak, et al., Am. J. Physiol, 269:G210-218 (1995); McLoed, et al., Gastroenterol., 106:405-413 (1994); Hochhaus, et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, ed., Bioreversible Carriers in Drug Design, American Pharmaceutical Association and Pergamon Press, 1987.

### Micronized Proton Pump Inhibitor

Particle size of the proton pump inhibitor can affect the solid dosage form in numerous ways. Since decreased particle size increases in surface area (S), the particle size reduction provides an increase in the rate of dissolution (dM/dt) as expressed in the Noyes-Whitney equation below:

dM/dt=dS/h(Cs−C)

M=mass of drug dissolved; t=time; D=diffusion coefficient of drug; S=effective surface area of drug particles; H=stationary layer thickness; Cs=concentration of solution at saturation; and C=concentration of solution at time t.

Because omeprazole, as well as other proton pump inhibitors, has poor water solubility, to aid the rapid dissolution of the drug product, various embodiments of the present invention use micronized omeprazole in the drug product formulation. In general, smaller particle size increases the bioabsorption rate of drug with substantially poor water solubility by increasing the surface area. In addition, small particle size also assists in maintaining better suspendibility since the smaller particles are less likely to “settle.” Thus, there is also a relationship between particle size and suspendibility.

Pharmaceutical formulations comprising micronized omeprazole are described herein. In some embodiments, the average particle size of at least about 90% the micronized omeprazole is less than about 100 μm, or less than about 80 μm, less than about 60 μm, or less than about 40 μm, or less than about 35 μm, or less than about 30 μm, or less than about 25 μm, or less than about 20 μm, or less than about 15 μm, or less than about 10 μm, or less than about 5 μm. In other embodiments, at least 80% of the micronized omeprazole has an average particle size of less than about 100 μm, or less than about 80 μm, less than about 60 μm, or less than about 40 μm, or less than about 35 μm, or less than about 30 μm, or less than about 25 μm, or less than about 20 μm, or less than about 15 μm, or less than about 10 μm, or less than about 5 μm. In still other embodiments, at least 70% of the micronized omeprazole has an average particle size less than about 100 μm, or less than about 80 μm, less than about 60 μm, or less than about 40 μm, or less than about 35 μm, or less than about 30 μm, or less than about 25 μm, or less than about 20 μm, or less than about 15 μm, or less than about 10 μm, or less than about 5 μm.

Pharmaceutical formulations wherein the micronized omeprazole is of a size which allows greater than 75% of the proton pump inhibitor to be released within about 1 hour, or within about 50 minutes, or within about 40 minutes, or within about 30 minutes, or within about 20 minutes, or within about 10 minutes or within about 5 minutes of dissolution testing are also provided herein. In some embodiments of the invention, the micronized omeprazole is of a size which allows greater than 90% of the proton pump inhibitor to be released within about 1 hour, or within about 50 minutes, or within about 40 minutes, or within about 30 minutes, or within about 20 minutes, or within about 10 minutes, or within about 5 minutes of dissolution testing.

### Antacids

The pharmaceutical composition of the invention comprises one or more antacid. A class of antacids useful in the present invention include, e.g., antacids possessing pharmacological activity as a weak base or a strong base. In one embodiment, the antacid, when formulated or delivered (e.g., before, during and/or after) with an proton pump inhibiting agent, functions to substantially prevent or inhibit the acid degradation of the proton pump inhibitor by gastrointestinal fluid for a period of time, e.g., for a period of time sufficient to preserve the bioavailability of the proton pump inhibitor administered.

In one aspect of the present invention, the antacid includes a salt of a Group IA metal, including, e.g., a bicarbonate salt of a Group IA metal, a carbonate salt of a Group IA metal, an alkali earth metal antacid, an aluminum antacid, a calcium antacid, or a magnesium antacid.

Other antacids suitable for the present invention include, e.g., alkali (sodium and potassium) or alkali earth (calcium and magnesium) carbonates, phosphates, bicarbonates, citrates, borates, acetates, phthalates, tartrate, succinates and the like, such as sodium or potassium phosphate, citrate, borate, acetate, bicarbonate and carbonate.

Pharmaceutical formulations comprising at least one antacid selected from an amino acid, an acid salt of an amino acid, an alkali salt of an amino acid, aluminum hydroxide, aluminum hydroxide/magnesium carbonate/calcium carbonate co-precipitate, aluminum magnesium hydroxide, aluminum hydroxide/magnesium hydroxide co-precipitate, aluminum hydroxide/sodium bicarbonate co-precipitate, aluminum glycinate, calcium acetate, calcium bicarbonate, calcium borate, calcium carbonate, calcium citrate, calcium gluconate, calcium glycerophosphate, calcium hydroxide, calcium lactate, calcium phthalate, calcium phosphate, calcium succinate, calcium tartrate, dibasic sodium phosphate, dipotassium hydrogen phosphate, dipotassium phosphate, disodium hydrogen phosphate, disodium succinate, dry aluminum hydroxide gel, L-arginine, magnesium acetate, magnesium aluminate, magnesium borate, magnesium bicarbonate, magnesium carbonate, magnesium citrate, magnesium gluconate, magnesium hydroxide, magnesium lactate, magnesium metasilicate aluminate, magnesium oxide, magnesium phthalate, magnesium phosphate, magnesium silicate, magnesium succinate, magnesium tartrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartrate, sodium acetate, sodium bicarbonate, sodium borate, sodium carbonate, sodium citrate, sodium gluconate, sodium hydrogen phosphate, sodium hydroxide, sodium lactate, sodium phthalate, sodium phosphate, sodium polyphosphate, sodium pyrophosphate, sodium sesquicarbonate, sodium succinate, sodium tartrate, sodium tripolyphosphate, synthetic hydrotalcite, tetrapotassium pyrophosphate, tetrasodium pyrophosphate, dipotassium phosphate, trisodium phosphate, and trometamol are provided herein. Based in part upon the list provided in The Merck Index, Merck & Co. Rahway, N.J. (2001).

In addition, due to the ability of proteins or protein hydrolysates to rapidly react with acids, they too can serve as antacids in the present invention. Furthermore, combinations of the above mentioned antacids can be used in the pharmaceutical formulations described herein.

The antacids useful in the present invention also include antacids or combinations of antacids that interact with HCl (or other acids in the environment of interest) faster than the proton pump inhibitor interacts with the same acids. When placed in a liquid phase, such as water, these antacids produce and maintain a pH greater than the pKa of the proton pump inhibitor.

Provided herein are pharmaceutical formulations wherein at least one antacid is selected from sodium bicarbonate, sodium carbonate, calcium carbonate, magnesium oxide, magnesium hydroxide, magnesium carbonate, aluminum hydroxide, and mixtures thereof. In one embodiment, the antacid is sodium bicarbonate and is present in about 0.1 mEq/mg proton pump inhibitor to about 5 mEq/mg proton pump inhibitor. In another embodiment, the antacid is a mixture of sodium bicarbonate and magnesium hydroxide, wherein the sodium bicarbonate and magnesium hydroxide are each present in about 0.1 mEq/mg proton pump inhibitor to about 5 mEq/mg proton pump inhibitor. In still another embodiment, the antacid is a mixture of sodium bicarbonate, calcium carbonate, and magnesium hydroxide, wherein the sodium bicarbonate, calcium carbonate, and magnesium hydroxide are each present in about 0.1 mEq/mg proton pump inhibitor to about 5 mEq/mg of the proton pump inhibitor.

Also provided herein are pharmaceutical formulations comprising at least one soluble antacid. Soluble antacids are useful for creating a for uniform suspension formation since insoluble antacids can settle over time if it does not form a colloidal suspension. For example, in one embodiment, the antacid is sodium bicarbonate and is present in about 0.1 mEq/mg proton pump inhibitor to about 5 mEq/mg proton pump inhibitor. In another embodiment, the antacid is a mixture of sodium bicarbonate and magnesium hydroxide, wherein the sodium bicarbonate and magnesium hydroxide are each present in about 0.1 mEq/mg proton pump inhibitor to about 5 mEq/mg proton pump inhibitor. The term “soluble antacid” as used herein refers to an antacid that has a solubility of at least 500 mg/mL, or 300 mg/mL, or 200 mg/mL, or 100 mL/mL in the gastrointestinal fluid.

In some embodiments of the present invention, the antacid is a specific particle size. For example, the average particle size of the antacid may be no greater than 20 μm, or no greater than 30 μm, or no greater than 40 μm, or no greater than 50 μm, or no greater than 60 μm, or no greater than 70 μm, or no greater than 80 μm, or no greater than 90 μm or no greater than 100 μm in diameter. In various embodiments, at least about 70% of the antacid is no greater than 20 μm, or no greater than 30 μm, or no greater than 40 μm, or no greater than 50 μm, or no greater than 60 μm, or no greater than 70 μm, or no greater than 80 μm, or no greater than 90 μm or no greater than 100 μm in diameter. In other embodiments, at least about 85% of the antacid is no greater than 20 μm, or no greater than 30 μm, or no greater than 40 μm, or no greater than 50 μm, or no greater than 60 μm, or no greater than 70 μm, or no greater than 80 μm, or no greater than 90 μm or no greater than 100 μm in diameter.

In various other embodiments of the present invention, the antacid is present in an amount of about 0.1 mEq/mg to about 5 mEq/mg of the proton pump inhibitor, or about 0.5 mEq/mg to about 3 mEq/mg of the proton pump inhibitor, or about 0.8 mEq/mg to about 2.5 mEq/mg of the proton pump inhibitor, or about 0.9 mEq/mg to about 2.0 mEq/mg of the proton pump inhibitor, or about 0.9 mEq/mg to about 1.8 mEq/mg of the proton pump inhibitor, or about 1.0 mEq/mg to about 1.5 mEq/mg of the proton pump inhibitor, or at least 0.5 mEq/mg of the proton pump inhibitor.

In another embodiment, the antacid is present in the pharmaceutical formulations of the present invention in an amount of about 0.1 mEq to about 15 mEq/mg of proton pump inhibitor, or about 0.1 mEq/mg of proton pump inhibitor, or about 0.5 mEq/mg of proton pump inhibitor, or about 1 mEq/mg of proton pump inhibitor, or about 2 mEq/mg of proton pump inhibitor, or about 2.5 mEq/mg of proton pump inhibitor, or about 3 mEq/mg of proton pump inhibitor, or about 3.5 mEq/mg of proton pump inhibitor, or about 4 mEq/mg of proton pump inhibitor, or about 4.5 mEq/mg of proton pump inhibitor, or about 5 mEq/mg of proton pump inhibitor, or about 6 mEq/mg of proton pump inhibitor, or about 7 mEq/mg of proton pump inhibitor, or about 8 mEq/mg of proton pump inhibitor, or about 9 mEq/mg of proton pump inhibitor, or about 10 mEq/mg of proton pump inhibitor, or about 15 mEq/mg of proton pump inhibitor.

In one embodiment, the antacid is present in the pharmaceutical formulations of the present invention in an amount of about 1 mEq to about 160 mEq per dose, or about 1 mEq, or about 5 mEq, or about 7 mEq, or about 10 mEq, or about 15 mEq, or about 20 mEq, or about 25 mEq, or about 30 mEq, or about 35 mEq, or about 40 mEq, or about 45 mEq, or about 50 mEq, or about 60 mEq, or about 70 mEq, or about 80 mEq, or about 90 mEq, or about 100 mEq, or about 110 mEq, or about 120 mEq, or about 130 mEq, or about 140 mEq, or about 150 mEq, or about 160 mEq per dose.

In another embodiment, the antacid is present in an amount of more than about 5 times, or more than about 10 times, or more than about 20 times, or more than about 30 times, or more than about 40 times, or more than about 50 times, or more than about 60 times, or more than about 70 times, or more than about 80 times, or more than about 90 times, or more than about 100 times the amount of the proton pump inhibiting agent on a weight to weight basis in the composition.

In another embodiment, the amount of antacid present in the pharmaceutical formulation is between 200 and 3500 mg. In other embodiments, the amount of antacid present in the pharmaceutical formulation is about 200 mgs, or about 300 mgs, or about 400 mgs, or about 500 mgs, or about 600 mgs, or about 700 mgs, or about 800 mgs, or about 900 mgs, or about 1000 mgs, or about 1100 mgs, or about 1200 mgs, or about 1300 mgs, or about 1400 mgs, or about 1500 mgs, or about 1600 mgs, or about 1700 mgs, or about 1800 mgs, or about 1900 mgs, or about 2000 mgs, or about 2100 mgs, or about 2200 mgs, or about 2300 mgs, or about 2400 mgs, or about 2500 mgs, or about 2600 mgs, or about 2700 mgs, or about 2800 mgs, or about 2900 mgs, or about 3000 mgs, or about 3200 mgs, or about 3500 mgs.

### Dosage

The proton pump inhibiting agent is administered and dosed in accordance with good medical practice, taking into account the clinical condition of the individual patient, the site and method of administration, scheduling of administration, and other factors known to medical practitioners. In human therapy, it is important to provide a dosage form that delivers the required therapeutic amount of the drug in vivo, and renders the drug bioavailable in a rapid manner. In addition to the dosage forms described herein, the dosage forms described by Phillips et al. in U.S. Pat. No. 6,489,346 are incorporated herein by reference.

The percent of intact drug that is absorbed into the bloodstream is not narrowly critical, as long as a therapeutic-disorder-effective amount, e.g., a gastrointestinal-disorder-effective amount of a proton pump inhibiting agent, is absorbed following administration of the pharmaceutical composition to a subject. It is understood that the amount of proton pump inhibiting agent and/or antacid that is administered to a subject is dependent on, e.g., the sex, general health, diet, and/or body weight of the subject.

Illustratively, administration of a substituted bicyclic aryl-imidazole to a young child or a small animal, such as a dog, a relatively low amount of the proton pump inhibitor, e.g., about 1 mg to about 30 mg, will often provide blood serum concentrations consistent with therapeutic effectiveness. Where the subject is an adult human or a large animal, such as a horse, achievement of a therapeutically effective blood serum concentration will require larger dosage units, e.g., about 10 mg, about 15 mg, about 20 mg, about 30 mg, about 40 mg, about 80 mg, or about 120 mg dose for an adult human, or about 150 mg, or about 200 mg, or about 400 mg, or about 800 mg, or about 1000 mg dose, or about 1500 mg dose, or about 2000 mg dose, or about 2500 mg dose, or about 3000 mg dose or about 3200 mg dose or about 3500 mg dose for an adult horse.

In various other embodiments of the present invention, the amount of proton pump inhibitor administered to a subject is, e.g., about 1-2 mg/Kg of body weight, or about 0.5 mg/Kg of body weight, or about 1 mg/Kg of body weight, or about 1.5 mg/Kg of body weight, or about 2 mg/Kg of body weight.

Treatment dosages generally may be titrated to optimize safety and efficacy. Typically, dosage-effect relationships from in vitro and/or in vivo tests initially can provide useful guidance on the proper doses for subject administration. Studies in animal models generally may be used for guidance regarding effective dosages for treatment of gastrointestinal disorders or diseases in accordance with the present invention. In terms of treatment protocols, it should be appreciated that the dosage to be administered will depend on several factors, including the particular agent that is administered, the route chosen for administration, the age of the subject, the condition of the particular subject.

In various embodiments, unit dosage forms for humans contain about 1 mg to about 120 mg, or about 1 mg, or about 5 mg, or about 10 mg, or about 15 mg, or about 20 mg, or about 30 mg, or about 40 mg, or about 50 mg, or about 60 mg, or about 70 mg, or about 80, mg, or about 90 mg, or about 100 mg, or about 110 mg, or about 120 mg of a proton pump inhibitor.

In a further embodiment of the present invention, the pharmaceutical formulation is administered in an amount to achieve a measurable serum concentration of a non-acid degraded proton pump inhibiting agent greater than about 100 ng/ml within about 30 minutes after administration of the pharmaceutical formulation. In another embodiment of the present invention, the pharmaceutical formulation is administered to the subject in an amount to achieve a measurable serum concentration of a non-acid degraded or non-acid reacted proton pump inhibiting agent greater than about 100 ng/ml within about 15 minutes after administration of the pharmaceutical formulation. In yet another embodiment, the pharmaceutical formulation is administered to the subject in an amount to achieve a measurable serum concentration of a non-acid degraded or non-acid reacted proton pump inhibiting agent greater than about 100 ng/ml within about 10 minutes after administration of the pharmaceutical formulation.

In another embodiment of the present invention, the composition is administered to the subject in an amount to achieve a measurable serum concentration of the proton pump inhibiting agent greater than about 150 ng/ml within about 15 minutes and to maintain a serum concentration of the proton pump inhibiting agent of greater than about 150 ng/ml from about 15 minutes to about 1 hour after administration of the composition. In yet another embodiment of the present invention, the composition is administered to the subject in an amount to achieve a measurable serum concentration of the proton pump inhibiting agent greater than about 250 ng/ml within about 1 hour and to maintain a serum concentration of the proton pump inhibiting agent of greater than about 150 ng/ml from about 15 minutes to about 1 hour after administration of the composition. In another embodiment of the present invention, the composition is administered to the subject in an amount to achieve a measurable serum concentration of the proton pump inhibiting agent greater than about 350 ng/ml within about 15 minutes and to maintain a serum concentration of the proton pump inhibiting agent of greater than about 150 ng/ml from about 15 minutes to about 1 hour after administration of the composition. In another embodiment of the present invention, the composition is administered to the subject in an amount to achieve a measurable serum concentration of the proton pump inhibiting agent greater than about 450 ng/ml within about 15 minutes and to maintain a serum concentration of the proton pump inhibiting agent of greater than about 150 ng/ml from about 15 minutes to about 1 hour after administration of the composition.

In another embodiment of the present invention, the composition is administered to the subject in an amount to achieve a measurable serum concentration of the proton pump inhibiting agent greater than about 150 ng/ml within about 30 minutes and to maintain a serum concentration of the proton pump inhibiting agent of greater than about 150 ng/ml from about 30 minutes to about 1 hour after administration of the composition. In yet another embodiment of the present invention, the composition is administered to the subject in an amount to achieve a measurable serum concentration of the proton pump inhibiting agent greater than about 250 ng/ml within about 30 minutes and to maintain a serum concentration of the proton pump inhibiting agent of greater than about 150 ng/ml from about 30 minutes to about 1 hour after administration of the composition. In another embodiment of the present invention, the composition is administered to the subject in an amount to achieve a measurable serum concentration of the proton pump inhibiting agent greater than about 350 ng/ml within about 30 minutes and to maintain a serum concentration of the proton pump inhibiting agent of greater than about 150 ng/ml from about 30 minutes to about 1 hour after administration of the composition. In another embodiment of the present invention, the composition is administered to the subject in an amount to achieve a measurable serum concentration of the proton pump inhibiting agent greater than about 450 ng/ml within about 30 minutes and to maintain a serum concentration of the proton pump inhibiting agent of greater than about 150 ng/ml from about 30 minutes to about 1 hour after administration of the composition.

In still another embodiment of the present invention, the composition is administered to the subject in an amount to achieve a measurable serum concentration of a non-acid degraded or non-acid reacted proton pump inhibiting agent greater than about 500 ng/ml within about 1 hour after administration of the composition. In yet another embodiment of the present invention, the composition is administered to the subject in an amount to achieve a measurable serum concentration of a non-acid degraded or non-acid reacted proton pump inhibiting agent greater than about 300 ng/ml within about 45 minutes after administration of the composition.

Contemplated compositions of the present invention provide a therapeutic effect as proton pump inhibiting agent medications over an interval of about 5 minutes to about 24 hours after administration, enabling, for example, once-a-day, twice-a-day, three times a day, etc. administration if desired.

Generally speaking, one will desire to administer an amount of the compound that is effective to achieve a serum level commensurate with the concentrations found to be effective in vivo for a period of time effective to elicit a therapeutic effect. Determination of these parameters is well within the skill of the art. These considerations are well known in the art and are described in standard textbooks.

In one embodiment of the present invention, the composition is administered to a subject in a gastrointestinal-disorder-effective amount, that is, the composition is administered in an amount that achieves a therapeutically-effective dose of a proton pump inhibiting agent in the blood serum of a subject for a period of time to elicit a desired therapeutic effect. Illustratively, in a fasting adult human (fasting for generally at least 10 hours) the composition is administered to achieve a therapeutically-effective dose of a proton pump inhibiting agent in the blood serum of a subject within about 45 minutes after administration of the composition. In another embodiment of the present invention, a therapeutically-effective dose of the proton pump inhibiting agent is achieved in the blood serum of a subject within about 30 minutes from the time of administration of the composition to the subject. In yet another embodiment, a therapeutically-effective dose of the proton pump inhibiting agent is achieved in the blood serum of a subject within about 20 minutes from the time of administration to the subject. In still another embodiment of the present invention, a therapeutically-effective dose of the proton pump inhibiting agent is achieved in the blood serum of a subject at about 15 minutes from the time of administration of the composition to the subject.

In further embodiments, greater than about 98%; or greater than about 95% of the drug absorbed into the bloodstream is in a non-acid degraded or a non-acid reacted form; or greater than about 90%; or greater than about 75%; or greater than about 50% of the drug absorbed into the bloodstream is in a non-acid degraded or a non-acid reacted form.

In other embodiments, the pharmaceutical formulations provide a release profile of the proton pump inhibitor, using USP dissolution methods, whereby greater than about 50% of the proton pump inhibitor is released from the composition within about 2 hours; or greater than about 70% of the proton pump inhibitor is released from the composition within about 2 hours; or greater than 50% of the proton pump inhibitor is released from the composition within about 1.5 hours; or greater than 50% of the proton pump inhibitor is released from the composition within about 1 hour after exposure to gastrointestinal fluid.

### Flavoring Agents

Proton pump inhibitors are inherently bitter tasting and in one embodiment of the present invention, one or more flavoring agents are used to make the bitter proton pump inhibitors more palatable. The “flavor leadership” criteria used to develop a palatable product include (1) immediate impact of identifying flavor, (2) rapid development of balanced, full flavor, (3) compatible mouth feel factors, (4) no “off” flavors, and (5) short aftertaste. See, e.g., Worthington, A Matter of Taste, Pharmaceutical Executive (April 2001). The pharmaceutical formulations of the present invention improve upon one or more of these criteria.

There are a number of known methods to determine the effect of a taste-masking material such as discrimination tests for testing differences between samples and for ranking a series of samples in order of a specific characteristic; scaling tests used for scoring the specific product attributes such as flavor and appearance; expert tasters used to both quantitatively and qualitatively evaluate a specific sample; affective tests for either measuring the response between two products, measuring the degree of like or dislike of a product or specific attribute, or determine the appropriateness of a specific attribute; and descriptive methods used in flavor profiling to provide objective description of a product are all methods used in the field.

Different sensory qualities of a pharmaceutical formulation such as aroma, flavor, character notes, and aftertaste can be measured using tests know in the art. See, e.g., Roy et al., Modifying Bitterness: Mechanism, Ingredients, and Applications (1997). For example, aftertaste of a product can be measured by using a time vs. intensity sensory measurement. And recently, modem assays have been developed to alert a processor of formulations to the bitter taste of certain substances. Using information known to one of ordinary skill in the art, one would readily be able-to determine whether one or more sensory quality of a pharmaceutical formulation of the present invention has been improved by the use of the taste-masking material.

Taste of a pharmaceutical formulation is important for both increasing patient compliance as well as for competing with other marketed products used for similar diseases, conditions and disorders. Taste, especially bitterness, is particularly important in pharmaceutical formulations for children since, because they cannot weigh the positive, getting better, against the immediate negative, the bitter taste in their mouth, they are more likely to refuse a drug that tastes bad. Thus, for pharmaceutical formulations for children, it becomes even more important to mask the bitter taste.

Flavoring agents useful in the pharmaceutical formulations of the present invention include, e.g., acacia syrup, acesulfame K, alitame, anise, apple, aspartame, neotame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, neotame, acesulfame potassium, mannitol, talin, sylitol, sucralose, sorbitol, swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof. In other embodiments, sodium chloride is incorporated into the pharmaceutical formulation.

Based on the proton pump inhibitor, antacid, suspension agent, and other excipients, as well as the amounts of each one, one of skill in the art would be able to determine the best combination of flavors to provide the optimally flavored product for consumer demand and compliance. See, e.g., Roy et al., Modifying Bitterness: Mechanism, Ingredients, and Applications (1997).

In other embodiments of the present invention, additional flavoring materials contemplated are those described in U.S. Pat. Nos. 4,851,226, 5,075,114, and 5,876,759. For further examples of taste-masking materials, see, e.g., Remington: The Science and Practice of Pharmacy, Nineteenth Ed. (Easton, Pa.: Mack Publishing Company, 1995); Hoover, John E., Remington's Pharmaceutical Sciences (Mack Publishing Co., Easton, Pa. 1975); Liberman, H. A. and Lachman, L., Eds., Pharmaceutical Dosage Forms (Marcel Decker, New York, N.Y., 1980); and Pharmaceutical Dosage Forms and Drug Delivery Systems, Seventh Ed. (Lippincott Williams & Wilkins 1999).

In another embodiment, the weight fraction of the flavoring agent is, e.g., about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the pharmaceutical composition.

In various embodiments of the invention, the total amount of flavoring agent present in the pharmaceutical formulations less than 20 grams, or less than 15 grams, or less than 10 grams, or less than 8 grams, or less than 5 grams, or less than 4 grams, or less than 3.5 grams, or less than 3 grams, or less than 2.5 grams or less than 2 grams, or less than 1.5 grams, or less than 1 gram, or less than 500 mg, or less than 250 mg, or less than 150 mg, or less than 100 mg, or less than 50 mg.

### Administration of Suspension

Suspensions can be used to supply drugs to the patient in liquid form. This type of formulation is especially important for patients who have difficulty swallowing solid dosage forms. The present invention provides a pharmaceutical formulation comprising at least one proton pump inhibitor, at least one antacid, at least one suspending agent, and at least one flavoring agent for oral administration in suspension by a subject.

In formulating the pharmaceutical formulations of the present invention, one of ordinary skill in the art will select excipients capable of producing and maintaining a homogeneous suspension. Two examples of general classes of excipients identified to yield homogeneous suspensions that do not easily ‘settle out’ over a short period of time, from the point of constitution to administration, are:
Suspending Agents: suspension homogeneity is provided by the suspending agent by increasing viscosity to reduce the settling of the suspended omeprazole particles; and/or
Wetting Agents: help with the initial wetting of the dry powder during constitution of the suspension and may also help prevent flocculation, or aggregation of particles in suspension.

Suspending agents contemplated for use in the present invention include, e.g., compounds such as polyvinylpyrrolidone, e.g., polyvinylpyrrolidone K12, polyvinylpyrrolidone K17, polyvinylpyrrolidone K25, or polyvinylpyrrolidone K30; polyethylene glycol, e.g., the polyethylene glycol can have a molecular weight of about 300 to about 6000, or about 3350 to about 4000, or about 7000 to about 5400; sodium alginate; gums, such as, e.g., gum tragacanth and gum acacia; guar gum; xanthans, including xanthan gum; sugars; cellulosics, such as, e.g., methylcellulose, sodium carboxymethylcellulose, hydroxypropylmethylcellulose, hydroxyethylcellulose; polysorbate-80; polyethoxylated sorbitan monolaurate; povidone; carageenan, Poloxamer F127; maltol; microcrystallline celluloses such as Avicel PH101 and Avicel CL-161; magnesium aluminum silicate, carbopol 974P; and the like.

Various embodiments of the present invention comprise at least about 2 mgs, or at least about 5 mgs, or at least about 7 mgs, or at least about 10 mgs, or at least about 13 mgs, or at least about 15 mgs, or at least about 20 mgs, or at least about 25 mgs, or at least about 30 mgs, or at least about 35 mgs, or at least about 40 mgs, or at least about 45 mgs, or at least about 50 mgs, or at least about 55 mgs, or at least about 60 mgs, or at least about 65 mgs, or at least about 70 mgs, or at least about 75 mgs, or at least about 80 mgs, or at least about 85 mgs, or at least about 90 mgs, or at least about 95 mgs, or at least about 100 mgs, or at least about 110 mgs, or at least about 120 mgs, or at least about 130 mgs, or at least about 140 mgs, or at least about 150 mgs of the suspending agent.

Provided herein are formulations wherein the suspending agent is a natural gum. In some embodiments, the suspending agent is xanthan gum or guar gum or gum Arabic (also known as Gum Acacia, Turkey Gum, Gum Senegal)

Wetting agents contemplated for use in the present invention include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, and the like.

Provided herein are pharmaceutical formulations wherein the dosage from is a powder for suspension, and upon admixture with water, a substantially uniform suspension is obtained. A suspension is “substantially uniform” when at least about 5 minutes after the pharmaceutical formulation is admixed with water, if suspension is split into equal top, middle and bottom sections from top to bottom, either:

(a) there is at least about 85% label claim of the proton pump inhibitor in each of the sections; and/or

(b) there is less than about 10% variation in the % label claim values among the sections.

In various embodiments of the present invention, flocculating agents are also used.

In some embodiments, a suspension is determined to be composed of approximately the same concentration of proton pump inhibitor throughout the suspension when there is less than about 25% to about 0.1%, or less than about 20% to about 1%, or less than about 15% to about 1%, or less than about 10% to about 1%, or less than about 25%, or less than about 20%, or less than about 15%, or less than about 13%, or less than about 11%, or less than about 9%, or less than about or 7%, less than about or 5%, or less than about 3%, or less than about 1%, or less than about 0.5%, or less than about 0.1% variation in concentration among samples taken from two or more points in the suspension.

In various embodiments, the amount of variation in proton pump inhibitor concentration among samples taken from various locations in the suspension is about 25%, or about 22.5%, or about 20%, or about 19%, or about 18%, or about 17%, or about 16%, or about 15%, or about 14%, or about 13%, or about 12%, or about 11%, or about 10%, or about 9%, or about 8%, or about 7%, or about 6%, or about 5%, or about 4%, or about 3%, or about 2%,—or about 1%, or about 0.5%, or about 0.1%.

The concentration at various points throughout the suspension can be determined by any suitable means known in the art, such as, e.g., methods described herein. For example, one suitable method of determining concentration at various points involves dividing the suspension into three substantially equal sections: top, middle and bottom. The layers are divided starting at the top of the suspension and ending at the bottom of the suspension. In other examples, any number of sections suitable for determining the uniformity of the suspension can be used, such as for example, two sections, three sections, four sections, five sections, or six or more sections. The sections can be named in any appropriate manner, such as relating to their location (e.g., top, middle, bottom), numbered (e.g., one, two, three, four, five, six, etc.), or lettered (e.g., A, B, C, D, E, F, G, etc.). The sections can be divided in any suitable configuration. In one embodiment, the sections are divided from top to bottom, which allows a comparison of sections from the top and sections from the bottom in order to determine whether and at what rate the proton pump inhibitor is settling into the bottom sections. A sample may be taken from each section with or without actual physical separation of the sections. Any number of the assigned sections suitable for determining uniformity of the suspension can be evaluated such as, e.g., all of the sections; 90% of the sections, 75% of the sections, 50% of the sections, 30% of the sections, or any other suitable number of sections.

Concentration is easily determined by methods known in the art. For example, concentration can be determined using percent label claim. “Percent label claim” (% label claim) is calculated using the actual amount of proton pump inhibitor per sample compared with the intended amount of proton pump inhibitor per sample. The intended amount of proton pump inhibitor per sample can be determined based on the formulation protocol or from any other suitable method, such as, for example, by referencing the “label claim,” that is, the intended amount of proton pump inhibitor depicted on labeling complying with the regulations promulgated by the United States Food and Drug Administration.

In one embodiment, the suspension is divided into sections and the percent label claim is determined for each section. In other embodiments, the suspension is determined to be substantially uniform if the suspension comprises at least about a set threshold percent label claim throughout the evaluated sections. The evaluated sections of the suspension can have any set threshold percent label claim suitable for determining that the suspension is substantially uniform. In various embodiments, the sections can comprise, e.g., at least about 70%, at least about 75%, at least about 80%, at least about 85%, at least about 87%, at least about 88%, at least about 89%, at least about 90%, at least about 93%, at least about 95%, at least about 98%, at least about 100%, at least about 105%, at least about 110%, or at least about 115% label claim of proton pump inhibitor, or any range that falls therein, such as, e.g., from about 80% to about 115%, from about 85% to about 110%, from about 87% to about 108%, from about 89% to about 106%, or from about 90% to about 105% label claim of proton pump inhibitor.

In some embodiments, 5 minutes after the pharmaceutical formulation is admixed with water, if the suspension is split, either physically or visually, into equal top, middle, and bottom sections from top to bottom, there is at least about 90%, or at least about 95%, or at least about 98% label claim of the proton pump inhibitor in each of the sections.

In one embodiment, at least about 10 minutes after the pharmaceutical formulation is admixed with water, if the suspension is split, either physically or visually, into equal top, middle, and bottom sections from top to bottom, there is at least about 80%, or at least about 85%; or at least about 87%, or at least about 90% label claim of the proton pump inhibitor in each of the sections. In another embodiment, at least about 15 minutes after the pharmaceutical formulation is admixed with water, if the-suspension is split into equal top, middle, and bottom sections from top to bottom, there is at least about 80%; or at least about 85%; or at least about 87%; or at least about 90% label claim of the proton pump inhibitor in each of the sections. In yet another embodiment, at least about 30 minutes after the pharmaceutical formulation is admixed with water, if the suspension is split, either physically or visually, into equal top, middle, and bottom sections from top to bottom, there is at least about 80%; or at least about 85%; or at least about 87%; or at least about 90% label claim of the proton pump inhibitor in each of the sections. In still other embodiments, at least about 45 minutes after the pharmaceutical formulation is admixed with water, if the suspension is split, either physically or visually, into equal top, middle, and bottom sections from top to bottom, there is at least about 80%; or at least about 85%; or at least about 87%; or at least about 90% label claim of the proton pump inhibitor in each of the sections. And, in still another embodiment, at least about 1 hour after the pharmaceutical formulation is admixed with water, if the suspension is split, either physically or visually, into equal top, middle, and bottom sections from top to bottom, there is at least about 70%; or at least about 80% or; at least about 85%; or at least about 87%; or at least about 90% label claim of the proton pump inhibitor in each of the sections. In other embodiments, at least about 2 hours after the pharmaceutical formulation is admixed with water, if the suspension is split, either physically or visually, into equal top, middle, and bottom sections from top to bottom, there is at least about 70%; or at least about 80% or; at least about 85%; or at least about 87%; or at least about 90% label claim of the proton pump inhibitor in each of the sections.

In other embodiments, the at least about 10 minutes after the pharmaceutical formulation is admixed with water, if the suspension is split, either physically or visually, into equal top, middle, and bottom sections from top to bottom, there is between about 85% to about 99% label claim of the proton pump inhibitor in each of the sections. In another embodiment, at least about 15 minutes after the pharmaceutical formulation is admixed with water, if the suspension is split, either physically or visually, into equal top, middle, and bottom sections from top to bottom, there is about 85% to about 99% label claim of the proton pump inhibitor in each of the sections. In yet another embodiment, at least about 30 minutes after the pharmaceutical formulation is admixed with water, if the suspension is split, either physically or visually, into equal top, middle, and bottom sections from top to bottom, there is about 85% to about 99% label claim of the proton pump inhibitor in each of the sections. In still another embodiment, at least about 45 minutes after the pharmaceutical formulation is admixed with water, if the suspension is split, either physically or visually, into equal top, middle, and bottom sections from top to bottom, there is about 85% to about 99% label claim of the proton pump inhibitor in each of the sections. In yet other embodiments, at least about 2 hours after the pharmaceutical formulation is admixed with water, if the suspension is split, either physically or visually, into equal top, middle, and bottom sections from top to bottom, there is about 85% to about 99% label claim of the proton pump inhibitor in each of the sections.

In another embodiment, the % label claim of the proton pump inhibitor in each of the sections remains substantially the same for up to about 5 minutes, or up to about 10 minutes, or up to about 15 minutes, or up to about 30 minutes, or up to about 45 minutes, or up to about 1 hour, or up to about 1.5 hours, or up to about 2 hours, or up to about 2.5 hours, or up to about 3 hours, or up to about 3.5 hours, or up to about 4 hours, or up to about 4.5 hours, or up to about 5 hours. The sections have remained “substantially the same” when the % label claim of the proton pump inhibitor has not changed by more than 10%.

In another embodiment, the % label claim of the proton pump inhibitor in each of the sections has not changed by more than about 20% for up to about 5 minutes, or up to about 10 minutes, or up to about 15 minutes, or up to about 30 minutes, or up to about 45 minutes, or up to about 1 hour, or up to about 1.5 hours, or up to about 2 hours, or up to about 2.5 hours, or up to about 3 hours, or up to about 3.5 hours, or up to about 4 hours, or up to about 4.5 hours, or up to about 5 hours.

In still other embodiments, the suspension is determined to be substantially uniform if the suspension comprises less than a set percentage variation in percent label claim throughout the evaluated sections. The evaluated sections of the suspension can have less than any set percentage variation in percent label claim suitable for determining that the suspension is substantially uniform such as, e.g., less than about 40%, less than about 35%, less than about 30%, less than about 25%, less than about 20%, less than about 17%, less than about 15%, less than about 13%, less than about 11%, less than about 10%, less than about 8%, less than about 5%, less than about 2%, or about 0% variation.

In some embodiments, at least about 5 minutes after the pharmaceutical formulation is admixed with water, if the suspension is split, either physically or visually, into equal top, middle, and bottom sections from top to bottom, there is less than about 10%, or less than about 8%, or less than about 5%, or less than about 3%, or less than about 1%, or less than about 0.1% variation in the % label claim values among the sections.

In one embodiment, at least about 10 minutes after the pharmaceutical formulation is admixed with water, if the suspension is split, either physically or visually, into equal top, middle, and bottom sections from top to bottom, there is less than about 20%; or less than about 15%, or less than about 12%; or less than about 10%; or less than about 8%; or less than about 5%; or less than about 2%, or less than about 1%, or less than about 0.5% variation, or less than about 0.3% variation, or less than about 0.1% variation in the % label claim values among the sections. In another embodiment, at least about 15 minutes after the pharmaceutical formulation is admixed with water, if the suspension is split, either physically or visually, into equal top, middle, and bottom sections from top to bottom there is less than about 20%, or less than about 15%; or less than about 12%; or less than about 10%; or less than about 5%; or less than about 2%, or less than about 1%, or less than about 0.5%, or less than about 0.3%, or less than about 0.1% variation in the % label claim values among the sections. In still another embodiment, at least about 30 minutes after the pharmaceutical formulation is admixed with water, if the suspension is split, either physically or visually, into equal top, middle, and bottom sections from top to bottom, there is less than about 20%, or less than about 15%; or less than about 12%; or less than about 10%; or less than about 5%; or less than about 2%, or less than about 1%, or less than about 0.5%, or less than about 0.3%, or less than about 0.1% variation in the % label claim values among the sections. In yet another embodiment, at least about 45 minutes after the pharmaceutical formulation is admixed with water, if the suspension is split, either physically or visually, into equal top, middle, and bottom sections from top to bottom, there is less than about 20%; or less than about 15%; or less than about 10%; or less than about 5%; or less than about 2%, or less than about 1%, or less than about 0.5%, or less than about 0.3%, or less than about 0.1% variation in the % label claim values among the sections. And in still other embodiments, at least about 1 hour after the pharmaceutical formulation is admixed with water, if the suspension is split, either physically or visually, into equal top, middle, and bottom sections from top to bottom, there is less than about 20%; or less than about 15%; or less than about 10%; or less than about 5%; or less than about 2%, or less than about 1%, or less than about 0.5%, or less than about 0.3%, or less than about 0.1% variation in the % label claim values among the sections.

In other embodiments of the present invention, there is less than about 10% variation in the % label claim values among the sections after at least 30 minutes, or after at least 1 hour, or after at least 1.5 hours, or after at least 2 hours, or after at least 2.5 hours, or after at least 3 hours, or after at least 3.5 hours, or after at least 4 hours, or after at least 4.5 hours, or after at least 5 hours.

Typically, the composition will remain substantially uniform for a suitable amount of time corresponding to the intended use of the composition. In various embodiments, the suitable amount of time corresponding to the intended use is, e.g., at least about 5 minutes, at least about 10 minutes, at least about 15 minutes, at least about 20 minutes, at least about 30 minutes, at least about 45 minutes, at least about 60 minutes, at least about 75 minutes, at least about 90 minutes, at least about 105 minutes, at least about 120 minutes, at least about 150 minutes, at least about 180 minutes, at least about 210 minutes, at least about 4 hours, at least about 5 hours, or greater than about 5 hours after admixture with water.

In one embodiment, the suspension remains substantially uniform from about 5 minutes to about 5 hours after admixture with water. In other embodiments, the suspension remains substantially uniform from at least about 15 minutes to about 45 minutes, from at least about 15 minutes to about 1.5 hours, from at least about 15 minutes to about 3 hours, from at least about 30 minutes to about 1 hour, from at least about 30 minutes to about 2 hours, from at least about 30 minutes to about 3 hours, from at least about 1 hour to about 2 hours, from at least about 1 to about 3 hours, and from at least about 1 hour to about 5 hours after admixture with water.

In one embodiment, the composition will remain substantially uniform at least until the suspension is prepared for administration to the patient. The suspension can be prepared for administration to the patient at any time after admixture as long as the suspension remains substantially uniform. In one embodiment, the suspension is prepared for administration to the patient from any time after admixture until the suspension is no longer uniform. For example, the suspension can be prepared for administration to the patient within about 5 minutes, within about 10 minutes, within about 15 minutes, within about 20 minutes, within about 30 minutes, within about 45 minutes, within about 60 minutes, within about 75 minutes, within about 90 minutes, within about 105 minutes, within about 120 minutes, within about 150 minutes, within about 180 minutes, within about 210 minutes, within about 4 hours, within about 5 hours, or more than about 5 hours after admixture with water.

In another embodiment, the suspension is prepared for administration to the patient from within about 5 minutes to about 2 hours after admixture. In still another embodiment, the suspension is prepared for administration to the patient from within about 15 minutes to about 1 hour after admixture. And in yet another embodiment, the suspension is prepared for administration to the patient within about 2 hours after admixture.

In some preferred embodiments, the pharmaceutical formulation comprises a gum suspending agent. In another embodiment, the composition comprises omeprazole, sodium bicarbonate and xanthan gum. In yet another embodiment, the composition comprises omeprazole, sodium bicarbonate, xanthan gum, and at least one flavoring agent.

In another embodiment, upon administration to a subject, the composition contacts the gastrointestinal fluid of the stomach and increases the gastrointestinal fluid pH of the stomach to a pH that prevents or inhibits acid degradation of the proton pump inhibiting agent in the gastrointestinal fluid of the stomach and allows a measurable serum concentration of the proton pump inhibiting agent to be absorbed into the blood serum of the subject, such that pharmacokinetic and pharmacodynamic parameters can be obtained using testing procedures known to those skilled in the art.

### Composition

The pharmaceutical formulations of the present invention contain desired amounts of proton pump inhibitor, antacid, suspending agent, and flavoring agent and can be in the form of, e.g., a powder such as a sterile packaged powder, a dispensable powder, and an effervescent powder. These pharmaceutical formulations of the present invention can be manufactured by conventional pharmacological techniques.

Conventional pharmacological techniques include, e.g., one or a combination of methods (1) dry mixing, (2) wet granulation (3) milling, and (4) dry or non-aqueous granulation. See, e.g., Lachman et al., The Theory and Practice of Industrial Pharmacy (1986). These methods, as well as other suitable methods, are known by one of ordinary skill in the art.

In one embodiment, the proton pump inhibitor is microencapsulated prior to being formulated into one of the above forms. In another embodiment, some or all of the antacid is also microencapsulated prior to being further formulated into one of the above forms. In some embodiments, the microencapsulation material is used to enhance the shelf-life of the pharmaceutical formulation. In other embodiments, the microencapsulation material is selected from cellulose hydroxypropyl ethers (HPC) such as Klucel®, Nisswo HPC and PrimaFlo HP22; low-substituted hydroxypropyl ethers (L-HPC); cellulose hydroxypropyl methyl ethers (HPMC) such as Seppifilm-LC, Pharmacoat®, Metolose SR, Opadry YS, PrimaFlo, MP3295A, Benecel MP824, and Benecel MP843; methylcellulose polymers such as Methocel® and Metolose®; Ethylcelluloses (EC) and mixtures thereof such as E461, Ethocel®, Aqualon®-EC, Surelease; Polyvinyl alcohol (PVA) such as Opadry AMB; hydroxyethylcelluloses such as Natrosol®; carboxymethylcelluloses and salts of carboxymethylcelluloses (CMC) such as Aualon®-CMC; polyvinyl alcohol and polyethylene glycol co-polymers such as Kollicoat IR®; monoglycerides (Myverol), triglycerides (KLX), polyethylene glycols, modified food starch, acrylic polymers and mixtures of acrylic polymers with cellulose ethers such as Eudragit® EPO, Eudragit® RD 100, and Eudragit® E100; cellulose acetate phthalate; sepifilms such as mixtures of HPMC and stearic acid, cyclodextrins, and mixtures of these materials. In still other embodiments, an antacid such as sodium bicarbonate is incorporated into the microencapsulation material. In another embodiment, an antioxidant is incorporated into the microencapsulation material. In yet another embodiment, a plasticizer is incorporated into the microencapsulation material.

In another embodiment, using standard coating procedures, such as those described in Remington's Pharmaceutical Sciences, 20th Edition (2000), a film coating is provided around the pharmaceutical formulation.

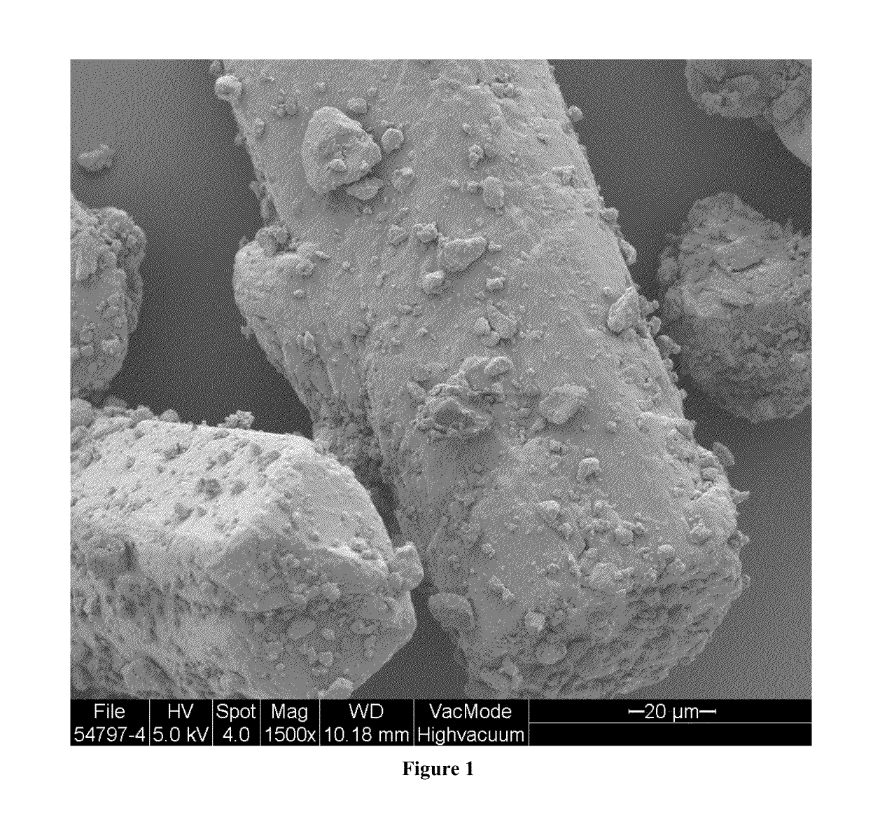

Pharmaceutical formulations comprising: (a) at least one acid-labile proton pump inhibitor in micronized form; and (b) at least one antacid, wherein the pharmaceutical formulation is made by a method comprising the steps of: (a) coating at least some of the at least one antacid with at least some of the micronized proton pump inhibitor to form a first blend; and (b) dry-blending the first blend with at least one other excipient are provided herein. The term “coating” refers to the process of contacting at least some of the micronized proton pump inhibitor to the surface of at least some of the antacid. Although the particles of antacid may be completely surrounded by the micronized omeprazole to form a “shell-like coating”, the use of the term “coating” is not intended to refer to only this instance. For example, in many instances the micronized omeprazole coats only part of the antacid, leaving some of the surface of the antacid particle uncoated. As shown in FIG. 1, micronized omeprazole or PPI can adhere to antacids. Although not wishing to be bound by theory, it is believed that the PPI adheres to the antacid via electrostatic or Van der Waals interaction. This transitioray coating can be pulled apart by external forces such as vacuum transfer of the “coated” material.

In other embodiments, the pharmaceutical formulations further comprise one or more additional materials such as a pharmaceutically compatible carrier, binder, filling agent, suspending agent, flavoring agent, sweetening agent, disintegrating agent, surfactant, preservative, lubricant, colorant, diluent, solubilizer, moistening agent, stabilizer, wetting agent, flocculating agent, anti-adherent, parietal cell activator, anti-foaming agent, antioxidant, chelating agent, antifungal agent, antibacterial agent, or one or more combination thereof.

(a) Particle Size

The particle size of the proton pump inhibitor, antacid and excipients is an important factor which can effect bioavailability, blend uniformity, segregation, and flow properties. In general, smaller particle sizes of a drug increases the bioabsorption rate of the drug with substantially poor water solubility by increasing the surface area. The particle size of the drug and excipients can also affect the suspension properties of the pharmaceutical formulation. For example, smaller particles are less likely to settle and therefore form better suspensions.

In various embodiments, the average particle size of the dry powder is less than about 500 microns in diameter, or less than about 450 microns in diameter, or less than about 400 microns in diameter, or less than about 350 microns in diameter, or less than about 300 microns in diameter, or less than about 250 microns in diameter, or less than about 200 microns in diameter, or less than about 150 microns in diameter, or less than about 100 microns in diameter, or less than about 75 microns in diameter, or less than about 50 microns in diameter, or less than about 25 microns in diameter, or less than about 15 microns in diameter. In other embodiments, the average particle size of the aggregates is between about 25 microns in diameter to about 300 microns in diameter. In still other embodiments, the average particle size of the aggregates is between about 25 microns in diameter to about 150 microns in diameter. And, in still further embodiments, the average particle size of the aggregates is between about 25 microns in diameter to about 100 microns in diameter. The term “average particle size” is intended to describe the average diameter of the particles and/or agglomerates used in the pharmaceutical formulation.

In another embodiment, the average particle size of the insoluble excipients is between about 5 μm to about 500 μm, or less than about 400 μm, or less than about 300 μm, or less than about 200 μm, or less than about 150 μm, or less than about 100 μm, or less than about 90 μm, or less than about 80 μm, or less than about 70 μm, or less than about 60 μm, or less than about 50 μm, or less than about 40 μm, or less than about 30 μm, or less than about 25 μm, or less than about 20 μm, or less than about 15 μm, or less than about 10 μm, or less than about 5 μm.

In other embodiments of the present invention, at least about 80% of the dry powder particles have a particle size of less than about 300 μm, or less than about 250 μm, or less than about 200 μm, or less than about 150 μm, or less than about 100 μm, or less than about 500 μm. In another embodiment, at least about 85% of the dry powder particles have a particle size of less than about 300 μm, or less than about 250 μm, or less than about 200 μm, or less than about 150 μm, or less than about 100 μm, or less than about 50 μm. In still other embodiments of the present invention, at least about 90% of the dry powder particles have a particle size of less than about 300 μm, or less than about 250 μm, or less than about 200 μm, or less than about 150 μm, or less than about 100 μm, or less than about 50 μm. In yet another embodiment, at least about 95% of the dry powder particles have a particle size of less than about 300 μm, or less than about 250 μm, or less than about 200 μm, or less than about 150 μm, or less than about 100 μm, or less than about 50 μm.

In another embodiment, the particle size of other excipients is chosen to be about the same as the particle size of the antacid. In yet another embodiment, the particle size of the insoluable excipients is chosen to be about the same as the particle size of the proton pump inhibitor.

Several factors can be considered in choosing both the proper excipient and its quantity. For example, the excipient should be pharmaceutically acceptable. Also, in some examples, rapid dissolution and neutralization of gastric acid to maintain the gastric pH at about 6.5 for at least one hour. The excipients which will be in contact with the proton pump inhibitor, if any, should also be chemically compatible with the proton pump inhibitor. “Chemically compatible” is intended to mean that the material does not lead to more than 10% degradation of the proton pump inhibitor when stored at room temperature for at least about 1 year.

Parietal cell activators are administered in an amount sufficient to produce the desired stimulatory effect without causing untoward side effects to patients. In one embodiment, the parietal cell activator is administered in an amount of about 5 mg to about 2.5 grams per 20 mg dose of the proton pump inhibitor.

(b) Exemplary Powder Compositions

Powders described herein can be prepared by mixing the proton pump inhibitor, one or more antacid, suspending agents, and pharmaceutical excipients to form a bulk blend composition. When referring to these bulk blend compositions as homogeneous, it is meant that the proton pump inhibitor, antacid, suspending agent, and excipients are dispersed evenly throughout the composition so that the composition may be readily subdivided into equally effective unit dosage forms. The individual unit dosages may also comprise film coatings, which disintegrate upon contact with diluent.

In various embodiments, the proton pump inhibitor, antacid, and optionally one or more excipients are dry blended and compressed into a mass, such as a tablet, having a hardness sufficient to provide a pharmaceutical composition that substantially disintegrates in water within less than about 5 minutes, less than about 10 minutes, less than about 20 minutes, less than about 30 minutes, less than about 40 minutes, less than about 50 minutes, or less than about 60 minutes. When at least 50% of the pharmaceutical composition has disintegrated, the compressed mass has substantially disintegrated.

A powder for suspension may be prepared by combining the micronized proton pump inhibitor, antacid, and suspending agent. In various embodiments, the powder may comprise one or more pharmaceutical excipients.

Effervescent powders are also prepared in accordance with the present invention. Effervescent salts have been used to disperse medicines in water for oral administration. Effervescent salts are granules or coarse powders containing a medicinal agent in a dry mixture, usually composed of sodium bicarbonate, citric acid and/or tartaric acid. When salts of the present invention are added to water, the acids and the base react to liberate carbon dioxide gas, thereby causing “effervescence.” Examples of effervescent salts include the following ingredients: sodium bicarbonate or a mixture of sodium bicarbonate and sodium carbonate, citric acid and/or tartaric acid. Any acid-base combination that results in the liberation of carbon dioxide can be used in place of the combination of sodium bicarbonate and citric and tartaric acids, as long as the ingredients were suitable for pharmaceutical use and result in a pH of about 6 or higher.

(c) Exemplary Solid Compositions

Solid compositions, e.g., tablets, chewable tablets, effervescent tablets, and capsules, are prepared by mixing the microencapsulated proton pump inhibitor with one or more antacid and pharmaceutical excipients to form a bulk blend composition. When referring to these bulk blend compositions as homogeneous, it is meant that the microencapsulated proton pump inhibitor and antacid are dispersed evenly throughout the composition so that the composition may be readily subdivided into equally effective unit dosage forms, such as tablets, pills, and capsules. The individual unit dosages may also comprise film coatings, which disintegrate upon oral ingestion or upon contact with diluent.

Compressed tablets are solid dosage forms prepared by compacting the bulk blend compositions described above. In various embodiments, compressed tablets of the present invention will comprise one or more flavoring agents. In other embodiments, the compressed tablets will comprise a film surrounding the final compressed tablet. In other embodiments, the compressed tablets comprise one or more excipients and/or flavoring agents.

A capsule may be prepared, e.g., by placing the bulk blend composition, described above, inside of a capsule.

A chewable tablet may be prepared by compacting bulk blend compositions, described above. In one embodiment, the chewable tablet comprises a material useful for enhancing the shelf-life of the pharmaceutical formulation. In another embodiment, microencapsulated material has taste-masking properties. In various other embodiments, the chewable tablet comprises one or more flavoring agents and one ore more taste-masking materials. In yet other embodiments the chewable tablet comprised both a material useful for enhancing the shelf-life of the pharmaceutical formulation and one or more flavoring agents.

In various embodiments, the microencapsulated proton pump inhibitor, antacid, and optionally one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the antacid and the proton pump inhibitor into the gastrointestinal fluid. When at least 50% of the pharmaceutical composition has disintegrated, the compressed mass has substantially disintegrated.

### Treatment

Initial treatment of a subject suffering from a disease, condition or disorder where treatment with an inhibitor of H⁺/K⁺-ATPase is indicated can begin with the dosages indicated above. Treatment is generally continued as necessary over a period of hours, days, or weeks to several months or years until the disease, condition or disorder has been controlled or eliminated. Subjects undergoing treatment with the compositions disclosed herein can be routinely monitored by any of the methods well known in the art to determine the effectiveness of therapy. Continuous analysis of such data permits modification of the treatment regimen during therapy so that optimal effective amounts of compounds of the present invention are administered at any point in time, and so that the duration of treatment can be determined as well. In this way, the treatment regimen/dosing schedule can be rationally modified over the course of therapy so that the lowest amount of an inhibitor of H⁺/K⁺-ATPase exhibiting satisfactory effectiveness is administered, and so that administration is continued only so long as is necessary to successfully treat the disease, condition or disorder.

In one embodiment, the pharmaceutical formulations are useful for treating a condition, disease or disorder where treatment with a proton pump inhibitor is indicated. In other embodiments, the treatment method comprises oral administration of one or more compositions of the present invention to a subject in need thereof in an amount effective at treating the condition, disease, disorder. In another embodiment, the disease, condition or disorder is a gastrointestinal disorder. The dosage regimen to prevent, give relief from, or ameliorate the disease, condition or disorder can be modified in accordance with a variety of factors. These factors include the type, age, weight, sex, diet, and medical condition of the subject and the severity of the disorder or disease. Thus, the dosage regimen actually employed can vary widely and therefore can deviate from the dosage regimens set forth herein.

In some embodiments, the pharmaceutical formulation is administered post meal. In further embodiments, the pharmaceutical formulation administered post meal is in the form of a chewable tablet.

The present invention also includes methods of treating, preventing, reversing, halting or slowing the progression of a gastrointestinal disorder once it becomes clinically evident, or treating the symptoms associated with, or related to the gastrointestinal disorder, by administering to the subject a composition of the present invention. The subject may already have a gastrointestinal disorder at the time of administration, or be at risk of developing a gastrointestinal disorder. The symptoms or conditions of a gastrointestinal disorder in a subject can be determined by one skilled in the art and are described in standard textbooks. The method comprises the oral administration a gastrointestinal-disorder-effective amount of one or more compositions of the present invention to a subject in need thereof.

Gastrointestinal disorders include, e.g., duodenal ulcer disease, gastrointestinal ulcer disease, gastroesophageal reflux disease, erosive esophagitis, poorly responsive symptomatic gastroesophageal reflux disease, pathological gastrointestinal hypersecretory disease, Zollinger Ellison Syndrome, and acid dyspepsia. In one embodiment of the present invention, the gastrointestinal disorder is heartburn.

Besides being useful for human treatment, the present invention is also useful for other subjects including veterinary animals, reptiles, birds, exotic animals and farm animals, including mammals, rodents, and the like. Mammals include primates, e.g., a monkey, or a lemur, horses, dogs, pigs, or cats. Rodents includes rats, mice, squirrels, or guinea pigs.

In various embodiments of the present invention, the compositions are designed to produce release of the proton pump inhibitor to the site of delivery (typically the stomach), while substantially preventing or inhibiting acid degradation of the proton pump inhibitor.

The present pharmaceutical compositions can also be used in combination (“combination therapy”) with another pharmaceutical agent that is indicated for treating or preventing a gastrointestinal disorder, such as, e.g., an anti-bacterial agent, an alginate, a prokinetic agent, a H2 antagonist, an antacid, or sucralfate, which are commonly administered to minimize the pain and/or complications related to this disorder.

Combination therapies contemplated by the present invention include administration of a pharmaceutical formulation of the present invention in conjunction with another pharmaceutically active agent that is indicated for treating or preventing a gastrointestinal disorder in a subject, as part of a specific treatment regimen intended to provide a beneficial effect from the co-action of these therapeutic agents for the treatment of a gastrointestinal disorder. The beneficial effect of the combination includes, but is not limited to, pharmacokinetic or pharmacodynamic co-action resulting from the combination of therapeutic agents. Administration of these therapeutic agents in combination typically is carried out over a defined time period (usually substantially simultaneously, minutes, hours, days, weeks, months or years depending upon the combination selected).

Combination therapies of the present invention are also intended to embrace administration of these therapeutic agents in a sequential manner, that is, where each therapeutic agent is administered at a different time, as well as administration of these therapeutic agents, or at least two of the therapeutic agents, in a substantially simultaneous manner. Substantially simultaneous administration can be accomplished, e.g., by administering to the subject a single tablet or capsule having a fixed ratio of each therapeutic agent or in multiple, single capsules, or tablets for each of the therapeutic agents. Sequential or substantially simultaneous administration of each therapeutic agent can be effected by any appropriate route.

The composition of the present invention can be administered orally or nasogastrointestinal, while the other therapeutic agent of the combination can be administered by any appropriate route for that particular agent, including, but not limited to, an oral route, a percutaneous route, an intravenous route, an intramuscular route, or by direct absorption through mucous membrane tissues. For example, the composition of the present invention is administered orally or nasogastrointestinal and the therapeutic agent of the combination may be administered orally, or percutaneously. The sequence in which the therapeutic agents are administered is not narrowly critical. Combination therapy also can embrace the administration of the therapeutic agents as described above in further combination with other biologically active ingredients, such as, but not limited to, a pain reliever, such as a steroidal or nonsteroidal anti-inflammatory drug, or an agent for improving stomach motility, e.g., and with non-drug therapies, such as, but not limited to, surgery.

The therapeutic compounds which make up the combination therapy may be a combined dosage form or in separate dosage forms intended for substantially simultaneous administration. The therapeutic compounds that make up the combination therapy may also be administered sequentially, with either therapeutic compound being administered by a regimen calling for two step administration. Thus, a regimen may call for sequential administration of the therapeutic compounds with spaced-apart administration of the separate, active agents. The time period between the multiple administration steps may range from, e.g., a few minutes to several hours to days, depending upon the properties of each therapeutic compound such as potency, solubility, bioavailability, plasma half-life and kinetic profile of the therapeutic compound, as well as depending upon the effect of food ingestion and the age and condition of the subject. Circadian variation of the target molecule concentration may also determine the optimal dose interval.

The therapeutic compounds of the combined therapies contemplated by the present invention, whether administered simultaneously, substantially simultaneously, or sequentially, may involve a regimen calling for administration of one therapeutic compound by oral route and another therapeutic compound by an oral route, a percutaneous route, an intravenous route, an intramuscular route, or by direct absorption through mucous membrane tissues, for example. Whether the therapeutic compounds of the combined therapy are administered orally, by inhalation spray, rectally, topically, buccally, sublingually, or parenterally (e.g., subcutaneous, intramuscular, intravenous and intradermal injections, or infusion techniques), separately or together, each such therapeutic compound will be contained in a suitable pharmaceutical formulation of pharmaceutically-acceptable excipients, diluents or other formulations components.

In one embodiment, the pharmaceutical formulations of the present invention are administered with low strength enteric coated Aspirin. In another embodiment, the second active pharmaceutical, e.g., Aspirin or an NSAID, used in combination with the pharmaceutical formulations of the present invention, is enteric coated. In other embodiments, antacid present in the pharmaceutical formulations of the present invention increase the pH level of the gastrointestinal fluid, thereby allowing part or all of the enteric coating on the second active pharmaceutical to dissolve in the stomach.

For the sake of brevity, all patents and other references cited herein are incorporated by reference in their entirety as if they appear in full within this document.

### EXAMPLES

The present invention is further illustrated by the following examples, which should not be construed as limiting in anyway. The experimental procedures to generate the data shown are discussed in more detail below. For all formulations herein, multiple doses may be proportionally compounded as is known in the art. The coatings, layers and encapsulations are applied in conventional ways using equipment customary for these purposes.

The invention has been described in an illustrative manner, and it is to be understood that the terminology used is intended to be in the nature of description rather than of limitation.

### Example 1

### Preparation of Omeprazole Plus Sodium Bicarbonate Powder for Suspension

This example demonstrates the preparation of omeprazole plus sodium bicarbonate powder for suspension (OSB-PFS). Each dosage of OSB-PFS contains omeprazole and sodium bicarbonate. The sodium bicarbonate in the OSB-PFS formulation protects the active ingredient omeprazole from acid degradation in vivo.

Various OSB-PFSs were formulated with the ingredients shown in Table 1 below:

TABLE 1
OSB-PFS Composition

Omeprazole
Sodium Bicarbonate
Sweetener(s)
Suspending Agent(s)
Flavoring Agent(s)

Illustrative OSB-PFS compositions comprising 20 mg of omeprazole are set forth in Table 2.

TABLE 2
Illustrative OSB-PFS Compositions (20 mg omeprazole)
Amounts in mg

| | 1 | 2 | 3 | 4 | 5 | 6 | 7 | 8 | 9 | 10 |
|---|---|---|---|---|---|---|---|---|---|---|
| Omeprazole | 20 | 20 | 20 | 20 | 20 | 20 | 20 | 20 | 20 | 20 |
| Sodium Bicarbonate | 1895 | 1680 | 1825 | 1895 | 1375 | 1650 | 1825 | 1650 | 1620 | 1600 |
| Xylitol 300 | 2000 | 2000 | 1500 | 1750 | 1750 | 2500 | 2000 | 1500 | 2000 | 2500 |
| (sweetener) | | | | | | | | | | |
| Sucrose-powder | 1750 | 2000 | 2250 | 2000 | 2500 | 1500 | 1750 | 2500 | 2000 | 1500 |
| (sweetener) | | | | | | | | | | |
| Sucralose (sweetener) | 125 | 100 | 150 | 75 | 100 | 70 | 80 | 130 | 125 | 80 |
| Xanthan Gum 75 | 17 | 55 | 31 | 80 | 39 | 48 | 72 | 25 | 64 | 68 |
| Peach Flavor | 47 | 15 | 75 | 32 | 60 | 50 | 77 | 38 | 35 | 62 |
| Peppermint | 26 | 10 | 29 | 28 | 36 | 42 | 56 | 17 | 16 | 50 |
| Total Weight | 5880 | 5880 | 5880 | 5880 | 5880 | 5880 | 5880 | 5880 | 5880 | 5880 |

Illustrative OSB-PFS compositions comprising 40 mg of omeprazole are set forth in Table 3.

TABLE 3
Illustrative OSB-PFS Compositions (40 mg omeprazole)
Amounts in mg

| | 1 | 2 | 3 | 4 | 5 | 6 | 7 | 8 | 9 | 10 |
|---|---|---|---|---|---|---|---|---|---|---|
| Omeprazole | 40 | 40 | 40 | 40 | 40 | 40 | 40 | 40 | 40 | 40 |
| Sodium Bicarbonate | 2010 | 1375 | 1680 | 1520 | 1400 | 1825 | 1680 | 1650 | 2030 | 1375 |
| Xylitol 300 | 1500 | 2750 | 2000 | 2500 | 2000 | 1750 | 2000 | 2500 | 1500 | 1750 |
| (sweetener) | | | | | | | | | | |
| Sucrose-powder | 2000 | 1500 | 2000 | 1500 | 2250 | 2000 | 2000 | 1500 | 2000 | 2500 |
| (sweetener) | | | | | | | | | | |
| Sucralose (sweetener) | 150 | 100 | 75 | 125 | 100 | 95 | 80 | 80 | 130 | 125 |
| Xanthan Gum 75 | 74 | 22 | 45 | 80 | 17 | 58 | 39 | 40 | 64 | 33 |
| Peach Flavor | 64 | 80 | 28 | 76 | 55 | 68 | 30 | 35 | 82 | 32 |
| Peppermint | 42 | 13 | 12 | 39 | 18 | 44 | 11 | 35 | 34 | 25 |
| Total Weight | 5880 | 5880 | 5880 | 5880 | 5880 | 5880 | 5880 | 5880 | 5880 | 5880 |

Illustrative OSB-PFS compositions comprising 60 mg of omeprazole are set forth in Table 4.

TABLE 4
Illustrative OSB-PFS Compositions (60 mg omeprazole)
Amounts in mg

| | 1 | 2 | 3 | 4 | 5 | 6 | 7 | 8 | 9 | 10 |
|---|---|---|---|---|---|---|---|---|---|---|
| Omeprazole | 60 | 60 | 60 | 60 | 60 | 60 | 60 | 60 | 60 | 60 |
| Sodium Bicarbonate | 1750 | 2475 | 1310 | 2130 | 2005 | 1580 | 1110 | 2300 | 1325 | 1400 |
| Xylitol 300 | 2000 | 1500 | 2000 | 1500 | 2000 | 2500 | 2250 | 1500 | 1750 | 2500 |
| (sweetener) | | | | | | | | | | |
| Sucrose-powder | 1750 | 1500 | 2250 | 2000 | 1500 | 1500 | 2250 | 1750 | 2500 | 1750 |
| (sweetener) | | | | | | | | | | |
| Sucralose (sweetener) | 145 | 130 | 75 | 70 | 150 | 150 | 60 | 100 | 80 | 75 |
| Xanthan Gum 75 | 15 | 57 | 22 | 19 | 64 | 39 | 33 | 29 | 44 | 50 |
| Peach Flavor | 92 | 105 | 87 | 78 | 57 | 31 | 69 | 95 | 88 | 25 |
| Peppermint | 68 | 53 | 76 | 23 | 44 | 20 | 48 | 46 | 33 | 20 |
| Total Weight | 5880 | 5880 | 5880 | 5880 | 5880 | 5880 | 5880 | 5880 | 5880 | 5880 |

Omeprazole powder, obtained from Union Quimico Farmaceutica S.A. (a.k.a. Uquifa), was micronized to a maximum diameter at 90% of 25 μm. Sodium bicarbonate grade (USP #1 grade) was chosen to complement the particle size of omeprazole in order to avoid stratification. Particle sizes of other excipients, such as the sweetener and suspending agent, were also carefully selected to achieve the maximum blend uniformity.

Omeprazole is a fluffy powder with a low bulk density while the major portion of the ingredients have a higher density and larger particle size. The content level of the active ingredient, omeprazole, was a relatively low percentage of the total weight. Geometric mixing of omeprazole with a suitable carrier assisted in distributing omeprazole evenly through the balance of the batch during the main mixing.

A flavor premixture was also implemented due to the extremely low density and cohesiveness of the flavor premix components. A small portion of sweetener was incorporated into the premixture. The material was then mixed for 15 minutes.

### Example II

### Exemplary Formulations Comprising Different Flavoring Agents

Omeprazole and omeprazole/bicarbonate suspensions were evaluated using the Flavor Profile Method of sensory analysis. The samples were evaluated according to the following protocol. Four-to-six trained professional sensory panelists participated in each panel session. All panelists tasted the same sample simultaneously. Panelists tasted no more than 3 ml of sample and the sample was held in the mouth for 10 seconds to provide time for evaluation and then the bulk of the sample was expectorated. There was a 20-minute washout period between samples during which panelists used spring water and unsalted crackers to rinse their mouths.

A variety of components were evaluated. Initial flavor and mouth feel attributes were recorded up to one minute. Aftertaste attributes were recorded at one, three, five, and ten minutes after expectoration.

Using this method the following flavor profiles were prepared for omeprazole in water (2 mg/ml).

| AROMA | | | | |
|---|---|---|---|---|
| Total Intensity of Aroma | 0 | | | |
| FLAVOR | | | | |
| Total Intensity of Flavor | 2 | | | |
| Bitter | 2 | | | |
| Sour | 1 | | | |
| Astringent | 1 | | | |
| Green Stemmy | 1.5 | | | |
| Waxy | 1 | | | |
| Tannin Mouthfeel | 1 | | | |
| Musty | 0.5 | | | |
| Salivating | 1 | | | |
| | 1 | 2 | 3 | 4 |
| AFTERTASTE | minute | minutes | minutes | minutes |
| Bitter | 2 | 2 | 1.5 | 1 |
| Sour | 1 | — | — | — |
| Astringent | 1 | — | — | — |
| Green Stemmy | 1.5 | 1.5 | 1.5 | 1 |
| Waxy | 1 | — | — | — |
| Tannin Mouthfeel | 1.5 | 1.5 | 1 | 1 |
| Salivating | 1 | 1.5 | — | — |

Using the same method described above, the following flavor profiles were prepared for omeprazole/sodium bicarbonate in water (2 mg/ml).

| AROMA | | | | |
|---|---|---|---|---|
| Total Intensity of Aroma | 0.5 | | | |
| Musty/Briny | 0.5 | | | |
| FLAVOR | | | | |
| Total Intensity of Flavor | 3 | | | |
| Salt | 1 | | | |
| Saline Mouthfeel | 1 | | | |
| Sour | 2 | | | |
| Bitter | 1.5 | | | |
| Metallic | 1.5 | | | |
| Fish amine-like | 2 | | | |
| Astringent | 1.5 | | | |
| Tannin Mouthfeel | 1.5 | | | |
| Tongue Sting | 1.5 | | | |
| Salivating | 1.5 | | | |
| | 1 | 2 | 3 | 4 |
| AFTERTASTE | minute | minutes | minutes | minutes |
| Bitter | 2 | 1 | 0.5 | — |
| Sour | 2 | 1.5 | 1 | 0.5 |
| Metallic | 1 | 1.5 | — | — |
| Fish amine-like | 1 | 1 | 0.5 | — |
| Tannin Mouthfeel | 1 | 1.5 | 1 | 1 |
| Tongue Sting | 1 | 1 | — | — |
| Salivating | 1.5 | 0.5 | — | — |

Once complete, various tablets comprising flavoring agents were made and tested using a similar method. Table 5 through table 11 illustrate 40 mg omeprazole tablets comprising different flavoring agents.

TABLE 5
OSB-PFS Compositions with Peach/Aspartame

| | Amount in mg |
|---|---|
| Omeprazole | 40 |
| Sodium Bicarbonate | 1680 |
| Calcium Phosphate | 100 |
| Guar Gum | 100 |
| Sucrose | 2000 |
| Xlitol, crystalline | 2000 |
| Aspartame | 250 |
| MagnaSweet 100 | 150 |
| Peppermint Flavor | 11 |
| Maltol | 20 |
| Peach Flavor | 60 |

TABLE 6
OSB-PFS Composition with Peach/Sucralose

| | Amount in mg |
|---|---|
| Omeprazole | 40 |
| Sodium Bicarbonate | 1680 |
| Calcium Phosphate | 100 |
| Guar Gum | 100 |
| Sucrose | 2000 |
| Xlitol, crystalline | 2000 |
| Sucralose | 40 |
| MagnaSweet 100 | 150 |
| Peppermint Flavor | 11 |
| Maltol | 20 |
| Peach Flavor | 60 |

TABLE 7
OSB-PFS Composition with Citrus Flavor/Sucralose

| | Amount mg |
|---|---|
| Omeprazole | 40 |
| Sodium Bicarbonate | 1680 |
| Calcium Phosphate | 100 |
| Guar Gum | 100 |
| Sucrose | 2000 |
| Xlitol, crystalline | 2000 |
| Sucralose | 40 |
| MagnaSweet 100 | 150 |
| Peppermint Flavor | 11 |
| Maltol | 20 |
| Peach Flavor | 60 |
| FNA lemon/lime flavor | 75 |

TABLE 8
OSB-PFS Composition with Citrus Flavor/Aspartame

| | Amount in mg |
|---|---|
| Omeprazole | 40 |
| Sodium Bicarbonate | 1680 |
| Calcium Phosphate | 100 |
| Guar Gum | 100 |
| Sucrose | 2000 |
| Xlitol, crystalline | 2000 |
| Aspartame | 250 |
| MagnaSweet 100 | 150 |
| Peppermint Flavor | 11 |
| Maltol | 20 |
| Peach Flavor | 60 |
| FNA lemon/lime flavor | 75 |

TABLE 9
OSB-PFS Composition with Red Fruit Flavor/Sucralose

| | Amount in mg |
|---|---|
| Omeprazole | 40 |
| Sodium Bicarbonate | 1680 |
| Calcium Phosphate | 100 |
| Guar Gum | 100 |
| Sucrose | 2000 |
| Xlitol, crystalline | 2000 |
| Sucralose | 40 |
| MagnaSweet 100 | 150 |
| Peppermint Flavor | 11 |
| Maltol | 20 |
| Peach Flavor | 60 |
| FNA Strawberry Flavor | 200 |
| FNA Cherry Flavor | 40 |

TABLE 10
OSB-PFS Composition with Red Fruit Flavor/Aspartame

| | Amount in mg |
|---|---|
| Omeprazole | 40 |
| Sodium Bicarbonate | 1680 |
| Calcium Phosphate | 100 |
| Guar Gum | 100 |
| Sucrose | 2000 |
| Xlitol, crystalline | 2000 |
| Aspartame | 250 |
| MagnaSweet 100 | 150 |
| Peppermint Flavor | 11 |
| Maltol | 20 |
| Peach Flavor | 60 |
| FNA Strawberry Flavor | 200 |
| FNA Cherry Flavor | 40 |

TABLE 11
OSB-PFS Composition with Peach/Sucralose

| | Amount in mg |
|---|---|
| Omeprazole | 40 |
| Sodium Bicarbonate | 1680 |
| Xanthan Gum | 390 |
| Sucrose | 2000 |
| Xlitol, crystalline | 2000 |
| Sucralose | 80 |
| Peppermint Flavor | 11 |
| Peach Flavor | 30 |

### Example III

### Omeprazole Plus Sodium Bicarbonate Powder for Suspension

The manufacture of the finished dosage form consisted of two separate processes: the manufacture of the ‘powder blend’ and the filling and packaging of the blend into individual packets using automated filling equipment.

The equipment used in the powder blending process was: 30 cu. ft. V-Blender for coating of micronized PPI to antacids, 4000 liter Scholl-Blender, automated vibrator sieve (equipped with #20 Mesh s/s), and a floor balance.

The powder blend was manufactured by the following steps:

a) The ingredients were weighed and screened through a 20 mesh screen and then dispensed into separate polyethylene bags:

b) Sodium bicarbonate and omeprazole were charged into a 30 cu. ft. V-shell Blender. The material was blended for 5 minutes. To this mixture, part of the Xylitol and Sucrose were loaded and the mixture was blended for 5 minutes. The omeprazole preblend was then discharged from the blender into a labeled container. This material was then passed through a #20 mesh s/s sieve into another labeled container. In some experiments, an automated vibrator sieve was used. Part of the sucrose, peppermint flavor, peach flavor, sucralose, and xanthan gum were then charged into the 5 cu. ft. V-shell Blender in the order listed above. This material was blended for 5 minutes.

After the material was blended, the flavor preblend was discharged from the blender into a labeled container and passed through a #20 mesh s/s sieve into a second labeled container. In one example, an automated vibrator sieve was used. Another part of the sucrose was then passed through a #20 mesh s/s sieve into a labeled container and another part of the xylitol was then passed through a #20 mesh s/s sieve into a labeled container. Automated vibrator sieve may be used.

The material was then divided into 2 equivalent portions. Part of the sodium bicarbonate was then passed through a #20 mesh s/s sieve again into a labeled container. The various preblends were then charged into a 4000 liter Scholl Blender and the material was then blended for 20 minutes. Once uniform, the final blend was discharged.

### Example VI

### Suspendability of Omeprazole Plus Sodium Bicarbonate Powder for Suspension

The example describes the determination of suspendability of omeprazole plus sodium bicarbonate powder for suspension with and without xanthan gum by HPLC. Both the physical and chemical testing results demonstrate that xanthan gum is needed as a suspending agent in the formulation.

A quantity of omeprazole sodium bicarbonate powder for suspension (40 mg) equivalent to 30 units was prepared by combining the appropriate amount of ingredients as described in Example 1.

Three sets of three separate samples were prepared with and without xanthan gum and assayed for content uniformity using an isocratic HPLC method with the following chromatographic parameters:

Column: 150 mm×3.9 mm with USP L7 (5 μm) packing
Guard Column: 20 mm×3.9 mm with USP L7 (5 μm) packing

### Detection: UV at 280 nm

### Column Temperature: Ambient

### Injection Volume: 20 μL

Flow Rate: 1 mL/min
Run Time: 15 minutes
Mobile Phase: 70:30 (v/v)=phosphate buffer, pH 7.0:acetonitrile
Sample Diluent: 75:25 (v/v)=10 mM sodium tetraborate borate:acetonitrile

The % label claim of omeprazole from each sampling position and for each individual sample was calculated. The mean values of % label claim and relative standard deviation (RSD) for each location and time point for the suspension samples prepared with and without Xanthan gum for each set of 3 samples are reported in Tables 12 and 13.

TABLE 12
Summary of Study Results for Suspendability Without Xanthan Gum

| | | Amount | % Label Claim | | | | | | | |
|---|---|---|---|---|---|---|---|---|---|---|
| | | of Sample | T = 5 minutes | | | | T = 1 hour | | | |
| | | Weighed | | | | Mean | | | | Mean |
| Set # | Sample # | (mg) | Top | Middle | Bottom | (RSD) | Top | Middle | Bottom | (RSD) |
| 1 | 1 | 5786 | 80.4 | 82.5 | 93.6 | 85.5 | 78.6 | 87.1 | 87.0 | 84.2 |
| | | | | | | (8.3) | | | | (5.8) |
| | 2 | 5903 | 77.1 | 77.6 | 88.4 | 81.0 | 71.6 | 70.0 | 95.7 | 79.1 |
| | | | | | | (7.9) | | | | (18.2) |
| | 3 | 5856 | 83.1 | 83.6 | 95.3 | 87.3 | 82.1 | 93.0 | 78.7 | 84.6 |
| | | | | | | (7.9) | | | | (8.8) |
| 2 | 1 | 5895 | 91.3 | 86.0 | 80.6 | 86.0 | 66.2 | 65.1 | 105.3 | 78.9 |
| | | | | | | (6.2) | | | | (29.0) |
| | 2 | 5866 | 81.9 | 85.7 | 92.1 | 86.6 | 58.4 | 60.2 | 109.7 | 76.1 |
| | | | | | | (6.0) | | | | (38.3) |
| | 3 | 5896 | 80.9 | 82.5 | 84.1 | 82.5 | 56.9 | 66.4 | 90.3 | 71.2 |
| | | | | | | (1.9) | | | | (24.2) |
| 3 | 1 | 5862 | 83.3 | 85.1 | 92.5 | 87.0 | 62.5 | 61.1 | 179.2 | 100.9 |
| | | | | | | (5.6) | | | | (67.2) |
| | 2 | 5865 | 82.6 | 85.4 | 94.3 | 87.4 | 44.9 | 57.5 | 123.3 | 75.2 |
| | | | | | | (7.0) | | | | (56.0) |
| | 3 | 5875 | 81.0 | 82.5 | 83.5 | 82.3 | 48.6 | 53.3 | 165.7 | 89.2 |
| | | | | | | (1.5) | | | | (74.3) |

TABLE 13
Summary of Study Results for Suspendability With Xanthan Gum

| | | Amount | % Label Claim | | | | | | | |
|---|---|---|---|---|---|---|---|---|---|---|
| | | of Sample | T = 5 minutes | | | | T = 1 hour | | | |
| | | Weighed | | | | Mean | | | | Mean |
| Set # | Sample # | (mg) | Top | Middle | Bottom | (RSD) | Top | Middle | Bottom | (RSD) |
| 1 | 1 | 5918 | 91.8 | 96.1 | 100.1 | 96.0 | 89.6 | 89.8 | 90.9 | 90.1 |
| | | | | | | (4.3) | | | | (0.8) |
| | 2 | 5901 | 91.7 | 96.1 | 102.7 | 96.8 | 90.6 | 90.7 | 89.4 | 90.2 |
| | | | | | | (5.7) | | | | (0.8) |
| | 3 | 5889 | 92.9 | 95.2 | 97.2 | 95.1 | 91.7 | 90.7 | 90.9 | 91.1 |
| | | | | | | (2.3) | | | | (0.6) |
| 2 | 1 | 5935 | 94.7 | 97.0 | 94.8 | 95.5 | 89.9 | 90.1 | 91.6 | 90.5 |
| | | | | | | (1.4) | | | | (1.0) |
| | 2 | 5891 | 95.0 | 95.1 | 94.1 | 94.7 | 91.0 | 90.4 | 91.2 | 90.9 |
| | | | | | | (0.6) | | | | (0.5) |
| | 3 | 5889 | 96.4 | 94.3 | 94.4 | 95.0 | 91.3 | 92.6 | 93.6 | 92.5 |
| | | | | | | (1.2) | | | | (1.2) |
| 3 | 1 | 5872 | 92.5 | 93.4 | 95.3 | 93.7 | 88.3 | 88.8 | 87.9 | 88.3 |
| | | | | | | (1.5) | | | | (0.5) |
| | 2 | 5876 | 94.8 | 95.0 | 94.7 | 94.8 | 91.9 | 92.2 | 92.9 | 92.3 |
| | | | | | | (0.2) | | | | (0.6) |
| | 3 | 5875 | 93.8 | 93.5 | 94.3 | 93.9 | 89.3 | 91.0 | 91.3 | 90.5 |
| | | | | | | (0.4) | | | | (1.2) |

The experiment was repeated and the mean values of % Label Claim and RSD for each location and time point for the suspension samples prepared with and without xanthan gum for each set of 3 samples are reported in Tables 14 and 15.

TABLE 14
Summary of Study Results for Suspendability Without Xanthan Gum

| | | Amount of | % Label Claim | | | | | | | |
|---|---|---|---|---|---|---|---|---|---|---|
| | | Sample | T = 5 minutes | | | | T = 1 hour | | | |
| | | Weighed | | | | Mean | | | | Mean |
| Set # | Sample # | (mg) | Top | Middle | Bottom | (RSD) | Top | Middle | Bottom | (RSD) |
| 1 | 1 | 5831 | 68.7 | 68.3 | 70.8 | 69.3 | 75.2 | 68.1 | 68.7 | 70.7 |
| | | | | | | (1.9) | | | | (5.6) |
| | 2 | 5830 | 66.1 | 61.9 | 61.9 | 63.3 | 65.0 | 66.3 | 65.4 | 65.6 |
| | | | | | | (3.8) | | | | (1.0) |
| | 3 | 5841 | 88.0 | 85.6 | 93.7 | 89.1 | 81.1 | 81.5 | 93.2 | 85.3 |
| | | | | | | (4.7) | | | | (8.1) |
| 2 | 1 | 5838 | 78.7 | 79.1 | 77.9 | 78.6 | 63.0 | 64.6 | 64.7 | 64.1 |
| | | | | | | (0.8) | | | | (1.5) |
| | 2 | 5834 | 81.4 | 84.4 | 84.7 | 83.5 | 73.6 | 64.0 | 57.1 | 64.9 |
| | | | | | | (2.2) | | | | (12.8) |
| | 3 | 5842 | 79.5 | 76.7 | 84.1 | 80.1 | 60.0 | 60.1 | 70.2 | 63.4 |
| | | | | | | (4.7) | | | | (9.2) |
| 3 | 1 | 5850 | 83.6 | 78.2 | 79.7 | 80.5 | 61.9 | 55.3 | 52.7 | 56.6 |
| | | | | | | (0.8) | | | | (8.4) |
| | 2 | 5841 | 73.5 | 70.3 | 66.9 | 70.2 | 57.4 | 45.1 | 45.3 | 49.3 |
| | | | | | | (4.7) | | | | (14.3) |
| | 3 | 5843 | 74.9 | 74.8 | 72.7 | 74.1 | 55.7 | 57.0 | 80.4 | 64.4 |
| | | | | | | (1.7) | | | | (21.6) |

TABLE 15
Summary of Study Results for Suspendability With Xanthan Gum

| | | Amount of | % Label Claim | | | | | | | |
|---|---|---|---|---|---|---|---|---|---|---|
| | | Sample | T = 5 minutes | | | | T = 1 hour | | | |
| | | Weighed | | | | Mean | | | | Mean |
| Set # | Sample # | (mg) | Top | Middle | Bottom | (RSD) | Top | Middle | Bottom | (RSD) |
| 1 | 1 | 5851 | 92.6 | 93.4 | 94.1 | 93.4 | 92.5 | 91.2 | 91.9 | 91.9 |
| | | | | | | (0.8) | | | | (0.7) |
| | 2 | 5887 | 92.9 | 95.5 | 95.8 | 94.7 | 92.3 | 90.7 | 90.1 | 91.0 |
| | | | | | | (1.7) | | | | (1.2) |
| | 3 | 5873 | 92.9 | 94.1 | 95.9 | 94.3 | 90.6 | 92.0 | 89.7 | 90.8 |
| | | | | | | (1.6) | | | | (1.3) |
| 2 | 1 | 5876 | 92.5 | 93.8 | 93.7 | 93.3 | 94.2 | 93.0 | 93.8 | 93.7 |
| | | | | | | (0.8) | | | | (0.7) |
| | 2 | 5869 | 94.8 | 94.8 | 95.3 | 95.0 | 94.1 | 95.1 | 94.2 | 94.5 |
| | | | | | | (0.3) | | | | (0.6) |
| | 3 | 5889 | 95.1 | 95.7 | 96.1 | 95.6 | 92.0 | 91.8 | 95.0 | 92.9 |
| | | | | | | (0.5) | | | | (1.9) |
| 3 | 1 | 5870 | 93.5 | 94.3 | 93.2 | 93.7 | 91.7 | 90.6 | 92.8 | 91.7 |
| | | | | | | (0.6) | | | | (1.2) |
| | 2 | 5871 | 92.0 | 93.4 | 93.1 | 92.8 | 92.1 | 92.5 | 92.4 | 92.3 |
| | | | | | | (0.8) | | | | (0.2) |
| | 3 | 5880 | 93.7 | 93.7 | 93.8 | 93.7 | 92.5 | 91.6 | 92.2 | 92.1 |
| | | | | | | (0.1) | | | | (0.5) |

These results show that in the presence of xanthan gum, satisfactory suspendability was observed by two separate analysts for up to 3 hours after constitution. In the absence of xanthan gum, suspendability results were poorer even after only 5 minutes following constitution and deteriorated during a period of standing of 3 hours. Visual observations showed that the suspension (white/off-white) without xanthan gum after 1 hour begins to precipitate and after three hours more precipitation was observed. The suspension with xanthan gum (off-white) showed no precipitation of the powder after 1 hour and 3 hours.

As demonstrated by Tables 12-15, the results show that in the absence of xanthan gum, suspendability was very poor and this conclusion was supported by visual observations.

### Example V

### Adherence of Omeprazole to Typical Administration Devices

This example demonstrates that the omeprazole portion of the OSB-PFS does not adhere to typical administration devices.

Ancillary devices used in the constitution and administration of the OSB-PFS may include dosing cups, syringes, and gastric sump tubes (nasogastric or orogastric tubes). A recovery study was conducted that investigated the adherence of OSB-PFS to gastric sump tubes. The in vitro study included passing 20 mL of constituted OSB-PFS through an 18 French gastric sump tube followed by a 20 mL water wash. The average omeprazole recovery for this study was greater than 90% omeprazole. Therefore, omeprazole does not significantly adhere to typical administration devices.

### Example VI

### Omeprazole Formulation and Excipients

In addition to those suspending and wetting agents described herein, other exemplary suspending and wetting agents are known in the art. See, e.g., Handbook of Pharmaceutical Excipients (2000). The following is a partial list of suspending and wetting agents with exemplary amounts:

| Functional Categories | Excipient Screened |
|---|---|
| Suspending Agents | Carageenan (0.05%-0.1%), |
| (w/w-suspension wt) | Xanthan Gum (0.05%-1.0%), |
| | Povidone K25 (0.1%-5.0%), |
| | Poloxamer F127 (0.05%-2.0%), |
| | Guar Gum (0.01%-1.0%), |
| | Maltol(1.0%-5.0%), |
| | Hydroxypropylmethylcellulose |
| | or HPMC (0.1%-5.0%) |
| | Avicel PH101 (0.05%-1.0%), |
| | Avicel CL-161 (0.05%-1.0%), |
| | Magnesium Aluminum Silicate (0.5%-2.0%), |
| | Carbopol 974P (0.5%-1.0%) |
| Wetting Agent | Sodium lauryl sulfate (0.025%) |
| (w/w-suspension wt) | |

To select a suitable suspending agent, experiments are conducted that measure the solubility of the suspending agent to determine the optimum concentration, its affect on suspendibility of omeprazole, and its impact on the chemical stability of omeprazole.

### Example VII

### Exemplary Excipients and Particle Sizes

As discussed herein, particle size of the materials is important to maintaining a suspension. The following are examples of excipients which could be used with a micronized proton pump inhibitor.

| Excipient | Particle size |
|---|---|
| Sodium Bicarbonate, USP #1 | 60% < 44 microns |
| Xylitol 300 | Mean = 150 micron |
| Sucrose, powdered | 94% < 75 microns |
| Sucralose | 90% ≦ 12 micron |
| Xanthan Gum | 95% ≦ 177 micron |
| Peach Flavor | 99% ≦ 840 micron |
| Peppermint Flavor | 99% ≦ 840 micron |
| Sodium Bicarbonate, USP #1 | Mean = 70 microns |
| Xylitol 300 | Mean = 150 micron |
| Sucrose, powdered | 94% < 75 microns |
| Sucralose | 90% ≦ 12 micron |
| Xanthan Gum | 95% ≦ 177 micron |
| Peach Flavor | 99% ≦ 840 micron |
| Peppermint Flavor | 99% ≦ 840 micron |
| Sodium Bicarbonate, USP #2 | Mean = 90 microns |
| Xylitol 300 | Mean = 150 micron |
| Sucrose, powdered | 94% < 75 microns |
| Sucralose | 90% ≦ 12 micron |
| Xanthan Gum | 95% ≦ 77 micron |
| Peach Flavor | 99% ≦ 840 micron |
| Peppermint Flavor | 99% ≦ 840 micron |
| Sodium Bicarbonate | 60% > 70 microns |
| Xylitol 300 | Mean = 150 micron |
| Sucrose, powdered | 94% < 75 microns |
| Sucralose | 90% ≦ 12 micron |
| Xanthan Gum | 95% ≦ 177 micron |
| Peach Flavor | 99% ≦ 840 micron |
| Peppermint Flavor | 99% ≦ 840 micron |
| Sodium Bicarbonate, USP #2 | 80% > 70 microns |
| Xylitol 300 | Mean = 150 micron |
| Sucrose, powdered | 94% < 75 microns |
| Sucralose | 90% ≦ 12 micron |
| Xanthan Gum | 95% ≦ 177 micron |
| Peach Flavor | 99% ≦ 840 micron |
| Peppermint Flavor | 99% ≦ 840 micron |
| Sodium Bicarbonate, USP #2 | 60% > 90 microns |
| Xylitol 300 | Mean = 150 micron |
| Sucrose, powdered | 94% < 75 microns |
| Sucralose | 90% ≦ 12 micron |
| Xanthan Gum | 95% ≦ 177 micron |
| Peach Flavor | 99% ≦ 840 micron |
| Peppermint Flavor | 99% ≦ 840 micron |

The invention has been described in an illustrative manner, and it is to be understood that the terminology used is intended to be in the nature of description rather than of limitation. All patents and other references cited herein are incorporated herein by reference in their entirety. Obviously, many modifications, equivalents, and variations of the present invention are possible in light of the above teachings. Therefore, it is to be understood that within the scope of the appended claims, the invention may be practiced other than as specifically described.

What is claimed:

1. A method of making a pharmaceutical formulation for oral administration comprising:
(a) making a first blend by dry blending at least one acid-labile substituted bicyclic aryl-imidazole proton pump inhibitor in micronized form with at least one bicarbonate salt of a Group IA metal, wherein (i) at least 80% of the micronized proton pump inhibitor is less than 40 μm in diameter; and (ii) the dry blending coats at least some of the bicarbonate salt with at least some of the micronized proton pump inhibitor; and
(b) blending the first blend with at least one other excipient comprising xanthan gum in an amount of about 50 mg to about 150 mgs.

2. The method according to claim 1, wherein said pharmaceutical formulation is a dosage form selected from a powder, a tablet, a bite-disintegration tablet, a chewable tablet, a caplet, a capsule, an effervescent powder, a rapid-disintegration tablet, and an aqueous suspension produced from a powder.

3. The method according to claim 1, wherein said proton pump inhibitor is selected from the group consisting of omeprazole, hydroxyomeprazole, esomeprazole, tenatoprazole, lansoprazole, pantoprazole, rabeprazole, dontoprazole, habeprazole, perprazole, ransoprazole, pariprazole, leminoprazole; or a free base, free acid, salt, hydrate, ester, amide, enantiomer, isomer, tautomer, and polymorph thereof.

4. The method according to claim 1, wherein said bicarbonate salt of the Group IA metal is a soluble antacid.

5. The method according to claim 4, wherein the soluble antacid is sodium bicarbonate.

6. The method according to claim 5, wherein the sodium bicarbonate is present in an amount of about 500 mgs to about 3000 mgs.

7. The method according to claim 2, wherein the pharmaceutical formulation is a powder or an aqueous suspension produced from a powder.

8. The method according to claim 1, wherein the average particle size of any substantially insoluble material in the pharmaceutical formulation is less than 150 μm in diameter.

9. The method according to claim 1, wherein the at least one acid-labile substituted bicyclic aryl-imidazole proton pump inhibitor is omeprazole.
